SEQUENCE LISTING (44 sequences)

SEQ ID NO: 1 - protein, 283 aa, unidentified
  REGION 1..283: note = Meiothermus silvanus
MPRFGAHAFIWAAEWNPEAAEKVIQGATRVGLDFVEIPLLHPESFDVELTRRLLNGYGVGCTCSLGLPREASLPEHPEAATRFLIQALNVAHQIGSEVLTGVTYATLGTLSGRAPREADYQAVVKALKPAARHAAALGMRLGIEPVNRYETYLINLAAQGLELIRRLDEPNVFLHLDTYHMNIEEKGFRGPIVEAGAHLGYIHLSESDRGTPGTGNVHWDAVFAGLREIGFSGDLVMESFVALNPDIARATCMWRDVVGDPQALVQEGLAFLRGKASEYGLLG

SEQ ID NO: 2 - DNA, 852 bp, unidentified
  misc_feature 1..852: note = Meiothermus silvanus
  source 1..852: mol_type = genomic DNA
atgccacgcttcggagctcatgcgttcatttgggccgctgagtggaacccggaagcggcagagaaagtgattcagggtgcaacccgtgttggcctcgacttcgttgaaattccgctcttgcacccggaatccttcgatgtcgagctcacacgtcgtttactaaacgggtacggcgtcggttgtacgtgttctctgggcttgccgcgtgaagcgtccctgccggagcaccccgaagccgcgactcgttttctgattcaggccctgaatgttgcacatcagattggctcagaggtgctgaccggggtaacctacgcgactttaggcactttatctggccgggcgccgcgcgaagctgattatcaagcagtcgtaaaagcactgaaaccggcggctagacatgcagcagcactgggtatgcgcctcggcattgaacccgttaatcgttatgaaacttatttaattaacctggccgctcagggcctggagttaatccgtcgtctggacgaaccgaatgtgttcctgcacctggatacgtaccacatgaatattgaagagaaaggtttccggggcccgatcgtggaagcgggcgcccacctggggtatattcatctgagcgagagtgatcgcggcacccctggaacgggcaacgtccattgggacgcggtgttcgcggggctgcgtgaaattggcttctcaggcgatttggtcatggaaagcttcgtcgcactgaaccctgatattgcgcgcgctacatgcatgtggcgggacgtcgttggcgatccccaggcgctggtccaggaaggcttagcatttctccgtggcaaggctagcgagtatggcctactcggttaa

SEQ ID NO: 3 - protein, 282 aa, unidentified
  REGION 1..282: note = hydrothermal vent metagenome
MEGFGVHTSMWTMSWDKKGAEYAVSQAVRYQMDFLEIALLSPLDVDAQHSRRLLEKNHMRAICSLGLPEGAWLSNNPQAGVEYLKIALEKTAEMGCEALSGVIYGGIGERTGFPPTEKELDNVVRALGEAASYAKSLGLLLGIEPVNRYESHLINTGRQSVEMIEKVGASNMFVHLDTYHMNIEEKGVAQGILDAREHIKYIHLSESDRGTPGAGTCDWDEIFAVLAAINFKGGLAMESFVNMPPEIAYGLSIWRPVAKSADEVMENGLPFLRNKARQYQLL

SEQ ID NO: 4 - DNA, 849 bp, unidentified
  misc_feature 1..849: note = hydrothermal vent metagenome
  source 1..849: mol_type = other DNA
atggaaggttttggagtgcacacgagcatgtggactatgagttgggataagaagggtgcggagtatgctgtaagccaggcggtgcgctatcagatggattttcttgaaatcgctctgctgagtccactggatgtcgatgcccagcatagtcgccgtctgctagagaagaaccacatgcgcgcgatttgcagccttgggttaccggaaggtgcgtggctcagtaataaccctcaggccggagtggaatacctgaagatcgccctggaaaagactgcggagatgggttgcgaagcgctgtccggcgtaatctacggcggcattggcgaaagaacaggttttccgccgacggagaaagagctggacaatgtcgtgcgcgcgctgggtgaagcggcatcttacgcaaaatcgttaggccttctgctcggtattgaaccggtgaaccggtatgagtctcatctgattaatacgggacgtcagagtgtggaaatgattgaaaaggtcggtgcctctaacatgtttgttcaccttgacacataccacatgaatattgaagaaaagggtgtggcacaagggattctggacgcgcgcgaacacataaaatatatccacctgtcagagtctgaccgagggactccgggcgcaggcacgtgtgactgggatgagattttcgcagtcctggccgcaattaactttaaaggcggcttagcgatggaatcgttcgtgaacatgccgccggaaattgcctatggtctgtccatttggcgtccggtggctaaatcggctgatgaagtcatggaaaacggcctgccgtttctacgcaacaaagcgcgccagtatcagctgctgtaa

SEQ ID NO: 5 - protein, 289 aa, unidentified
  REGION 1..289: note = Thermoclostridium caenicola
MKYGIFYAYWEKEWKGDFITYIEKVKKLGFDILEVGCGDFHKQPDSYFHTLRDAAREYDIILTGGYGPRAEHNLCSPDTAVVENALAFYSDIFRKMEIAGIRSIGGGLYAYWPVDYSREPDKAGDLERSIKNMRRLADIAERHGITLNMEVLNRFEGYLINDTNEGLAYIRAVDKPNVKLMLDTFHMNIEEDSFTEPILQAGKYLGHVHVGEPNRKPPREGRIPWGEIGKALRQIGYDGPVVMEPFVTMGGQVGKDICVWRDLSQGATEEDLDRDAEKSLAFLKGMFEA

SEQ ID NO: 6 - protein, 870 aa, unidentified
  REGION 1..870: note = Thermoclostridium caenicola
ATGAAATATGGTATATTTTACGCATATTGGGAGAAAGAATGGAAAGGCGACTTTATCACATACATTGAGAAAGTTAAGAAATTAGGCTTTGACATTTTGGAAGTCGGCTGCGGTGATTTTCATAAACAGCCGGATTCATACTTCCACACCCTGCGTGATGCCGCTCGCGAATACGACATTATTCTGACCGGCGGCTATGGGCCGCGCGCCGAACACAACTTGTGTAGCCCGGATACAGCGGTCGTTGAAAATGCCCTGGCATTTTACTCCGATATATTTCGCAAAATGGAAATTGCCGGCATCCGTTCGATCGGCGGCGGTCTATATGCGTATTGGCCAGTTGATTACAGCCGTGAACCCGACAAAGCCGGCGATTTAGAACGTTCCATTAAGAACATGCGTCGCTTAGCCGATATTGCGGAACGGCATGGTATCACGCTGAATATGGAAGTGCTCAATCGCTTCGAAGGTTACCTTATTAATGATACCAATGAAGGTCTGGCCTACATTCGTGCCGTCGATAAACCCAACGTAAAGTTGATGCTGGATACATTTCACATGAACATTGAGGAAGATAGCTTTACCGAACCCATTCTCCAGGCTGGTAAGTACCTGGGCCATGTACATGTCGGCGAACCAAATCGTAAACCACCACGGGAAGGTCGTATTCCGTGGGGCGAGATTGGCAAAGCGCTGCGCCAGATTGGGTACGACGGTCCCGTGGTGATGGAACCGTTTGTTACCATGGGCGGCCAAGTGGGGAAAGACATTTGTGTGTGGCGGGACCTTAGCCAAGGTGCGACCGAAGAAGATCTGGATCGCGATGCTGAGAAAAGCCTGGCCTTTCTGAAAGGAATGTTTGAAGCCTAA

SEQ ID NO: 7 - protein, 290 aa, unidentified
  REGION 1..290: note = hydrothermal vent metagenome
MNLPAKKMVLGVHTFAVAPFWDLEVMRHEARRLKSHGVGLLEIPLLRPEEINIKATRAFAREFGFELVTSLGLPSNIDAVEDPQSALAFLEPAFKVAAEIGSNMLSGVTYAPIGKISGQPVTQREKDGICRFLQQAAALAANHGLKLGVEPCNRYETHLMNTAAQAVEYVEAVAAENLFIHLDTYHMNIEEESFAAGFAKAAPYLGYVHLSESNRGVPGRAMINWDAVMGALADIGYHGALTLESMNYVDPDIATALAVWRPVAKNPDDVIDFGLAFLLKAAADAGLTFG

SEQ ID NO: 8 - DNA, 873 bp, unidentified
  misc_feature 1..873: note = hydrothermal vent metagenome
  source 1..873: mol_type = other DNA
atgaacctgccggctaagaagatggtcctgggagtccatacgttcgcagtcgccccgttttgggatcttgaagtaatgcgccacgaggcccgccgtttaaagagccatggcgtagggttattggaaatcccacttctccggccagaagaaattaatattaaagcgacccgcgcatttgctcgtgaatttgggtttgaattagtgaccagtttaggcctgccttcgaatatcgacgctgtagaagatccgcagtctgctttagcctttctggaaccggcgttcaaagtggctgctgaaattggtagcaatatgctttcaggcgttacctatgctccgattggaaagataagtggtcagccggtgacccagcgtgagaaggatggtatttgtcggttcctacaacaagcggctgcgttggccgcgaaccatgggttgaaactgggcgtagagccttgtaatcgctatgaaacgcatctgatgaacacagcagcccaagcagttgaatacgttgaggctgtggcagcggaaaaccttttcatccacttagatacctaccacatgaacatcgaagaagaaagttttgcagcagggtttgcgaaagcggcgccttatctggggtacgtgcatctgagcgagagcaatcgcggcgtaccgggccgcgccatgatcaattgggatgcagtgatgggtgcccttgccgacatcgggtatcatggggcactgactctggagtctatgaattatgtggatccggatattgcgactgcccttgcggtctggcgtccggttgccaagaacccggacgatgtgatagactttggcctggcgtttctgctgaaggccgcagcggatgcgggtttgacattcggttaa

SEQ ID NO: 9 - protein, 282 aa, unidentified
  REGION 1..282: note = Meiothermus granaticius
MARFGAHAFIWSADWTPQAAEKVAAGAAAAGLDFVEIPLLRPEAFDSTLTRRLLEHHGLGCTCSLGLPTEAALPDHPQAAARFLIQALDVAHQMGSPVLSGVTYATLGALSGRPPTEADYETLAKTLKPVAQHAARLGMRLGLEPVNRYETYLINLGSQALDLIQRIGEPNVFVHLDTYHMNIEEKGFKNPIVTVGKHLGYIHLSESDRGTPGSGNVHWDEVFSGLQAIGFQGDLVMESFVALNPDIARATCMWRDVVGDPKTLVHDGLAFLRGKAREYGLL

SEQ ID NO: 10 - DNA, 849 bp, unidentified
  misc_feature 1..849: note = Meiothermus granaticius
  source 1..849: mol_type = genomic DNA
atggcgcgctttggtgcgcatgcttttatttggagtgctgattggacccctcaagccgccgaaaaggtcgcagcgggagcagcggcggcgggactggattttgtcgaaataccgcttcttcgtccagaggcgttcgattcgacacttacccgtaggttattagagcatcatggactgggatgcacctgctctctgggtctgcctaccgaggcagcactcccggaccatcctcaagcagcagcccgttttctcatccaggccttggatgtagcccatcaaatgggcagcccggtcctgagtggggtcacctacgcaaccctgggcgcactgtcaggccgcccgccgaccgaggccgattatgaaaccctggcgaaaacactgaagccagtagcgcagcacgccgcccgcctgggtatgcgactcgggcttgaaccggttaaccgctatgaaacatacctgataaacttagggtcacaggcgctggatctcatccaacgtatcggcgaaccgaacgtgttcgtacacctggatacttatcacatgaacatcgaagagaaaggttttaagaacccgattgttaccgtcggtaaacatcttggttatattcacctgtctgaaagcgaccgtggtaccccaggtagcggtaatgtgcattgggatgaagtgttctccggcctgcaagcaatcggttttcagggtgaccttgtgatggagtcgtttgtggcactgaatccggatattgcccgcgccacgtgcatgtggcgggatgttgtgggagatccgaaaacactggtgcatgatggcctagctttcctgcgcggtaaagctcgcgaatacggactgctgtaa

SEQ ID NO: 11 - protein, 283 aa, unidentified
  REGION 1..283: note = Rubellimicrobium thermophilum
MQGFGVHTSMWTMHWDRAGAERTIPAAAAYKMDFIEIALLDTAIVDAAHTRALLEKHGLRAVCSLGLPEPVWASVNPEGAIAHLKRALDKTAEMGAEALSGVTYGGIGQRTGVPPTPQEYDNIARALEAAAKHAKALGLAFGIEPVNRYENHLINTGRQAVEMIEKVGADNIFIHLDTYHMNIEEKGVANGILDAREHLRYIHLSESDRGTPGEGTCDWDEIFAALAAIGFKGGLAMESFINMPPQVAYGLAVWRPVAESFEEVMDRGLPFLRNKARQYRLIA

SEQ ID NO: 12 - DNA, 852 bp, unidentified
  misc_feature 1..852: note = Rubellimicrobium thermophilum
  source 1..852: mol_type = genomic DNA
atgcaaggtttcggcgtacacactagcatgtggacgatgcattgggatcgcgcgggcgccgagcgcaccattccggccgccgccgcctacaaaatggactttatcgaaattgcgctgctggatacagctatagtcgatgcagcgcacacccgtgcgctgctggagaagcacggcctgcgtgcagtttgttcactcggattaccggagccggtctgggcctctgtgaacccggaaggggccattgctcacctgaaacgcgcgctggacaagaccgcagagatgggagctgaagctctgtctggtgtgacttacggcggtatcggccagcgcacaggcgtaccaccaacgccccaagaatacgataacattgcccgtgcccttgaagcagctgcgaaacatgccaaagccttaggcctggcctttggtatcgaaccggttaacaggtatgaaaaccatttgataaacaccggacgtcaagctgtagaaatgatcgaaaaggtcggggcagataacattttcatacacctggatacgtatcacatgaatatcgaagagaaaggtgtggcaaacggtatcctggatgcccgagaacaccttcgttatattcacctgtcagaaagcgatcgcggcactcctggcgaagggacgtgtgattgggacgaaatcttcgctgcgttggccgccatcggttttaaaggcggccttgctatggaatcctttattaacatgccgcctcaagtcgcttacggcctggcggtgtggcgtcctgttgccgaatcttttgaagaagtcatggaccgtggcctgccgtttctacgtaacaaggctcgtcagtatagattgatcgcttaa

SEQ ID NO: 13 - protein, 282 aa, unidentified
  REGION 1..282: note = Candidatus Thermofonsia Clade 1 bacterium
MPTFGAHAFVWIGDWTTESGNYAIAQAGALGFDFIEIPLLAPQRFDAASHRQALAQAGIQATCSLVLPKGAHMPRYPERARQFLYEALEKVEAVGSQYLGGCIAYELGYLTGQPPTPEERQVVVEVLRDVAAEARRRGIQLALEACNRYETYLYNTLADVRETVLAVGAPNLKLHADTYHMNIEEEGFAQPLIACADVLDYIHMSESHRGLVGSGNVNWAQVWQALAAIRFNGKLVLESFAAINPDLQAATCLWRPPNQPPEVLAREGLRFLREGAAQAQLP

SEQ ID NO: 14 - DNA, 849 bp, unidentified
  misc_feature 1..849: note = Candidatus Thermofonsia Clade 1 bacterium
  source 1..849: mol_type = genomic DNA
atgccaaccttcggcgcccatgcatttgtgtggattggagattggacgacagaatcgggaaactatgcgatcgcgcaagccggcgcactgggcttcgacttcattgagattccgcttttggcaccgcagcgttttgatgccgcttcccaccgtcaagccctggcgcaggccggcattcaggcgacgtgcagcctggtattgccaaaaggcgctcacatgcccaggtatccagaacgtgcgcgtcagttcttgtatgaagcgttagaaaaggtagaggcggttggaagtcaatacttgggcggttgcatcgcgtacgaacttgggtatcttaccggtcagccaccgaccccggaagagcgtcaggttgttgtggaagtgctgcgcgatgtagccgcagaagcacgccgtcgcggcattcagttggcactggaagcatgcaatcgctatgagacttacctgtacaacacgctggcggacgtgcgcgaaacggttttagcggtgggcgcgccaaacttgaagctgcatgcggatacctaccacatgaacatcgaagaagaaggctttgcccagccgcttattgcctgtgccgacgtgctggattatatccacatgtcggaatcccatcgcggcctggtcggaagcggcaacgttaattgggcgcaggtctggcaagcgctcgccgcaattcgcttcaatggcaaactggtgctggaatcgtttgccgcgattaacccggaccttcaggccgctacttgtctgtggcgcccgcccaaccagccgccggaagtgctggcccgcgaaggactgcgatttctccgagaaggtgcggcacaggcccaactaccgtaa

SEQ ID NO: 15 - protein, 282 aa, unidentified
  REGION 1..282: note = Deinococcus koreensis
MLKFGAHAFCWEGDWTDEIGDRVIEQAARAGLDFIEIPLLHPETFDARRHRRHLEAVGLACVSSLGLPRDAHMPHEPEKAVTFLTGVLDRMEELGARDLTGCTGYSIGVLTGQGPTSQELDRMVDGLARVTEDARSRGIGVGLEAINRYETYMVNTLDDALAVVNRVGSDNLRVHADTYHMNIEETNLREALGRVKGKLNFIHMSESHRGLVGTGTVPWEDVWQGLADIEFSGYLTLESFAAPNAELAAATCIWKPPRHSGQELAQGGLAFLREGATRHGLM

SEQ ID NO: 16 - DNA, 849 bp, unidentified
  misc_feature 1..849: note = Deinococcus koreensis
  source 1..849: mol_type = genomic DNA
atgttgaagttcggcgcgcacgccttttgctgggaaggcgattggaccgatgaaattggcgatcgcgtcatcgagcaagcggcgcgggccggattggatttcattgaaatcccgctcttgcaccctgaaaccttcgatgcgcgccgtcatcgtcgccatcttgaggctgtgggcctggcgtgcgtttccagcctgggccttccgcgtgacgcccacatgccacatgaaccggagaaagcggtaacattccttaccggcgtactggatcgcatggaagaactgggcgctcgcgaccttaccggttgtaccggttatagcattggcgtactgaccggccagggtccgacctctcaagagctcgatcgtatggtggatggtctggctcgtgtgactgaagatgcacggtcgcgcgggattggcgtaggccttgaagccatcaatcgttatgaaacttatatggtaaacacgctggacgatgcgttagcggtcgtgaatcgtgtcgggagtgacaatcttcgcgttcatgccgatacataccacatgaacatagaagaaaccaacctgcgtgaggcgcttgggcgagttaagggtaagctgaacttcattcacatgagtgaaagtcatcgtggtctggttggtaccgggacagttccatgggaagatgtttggcagggtctggcggatattgagttcagcgggtatcttactcttgaatcgtttgcagctccgaatgccgagttggcggcagctacctgtatctggaaaccaccacggcatagcggccaggaactggcgcagggtggcctagcttttctgcgcgagggcgcgactcgccacggcttaatgtaa

SEQ ID NO: 17 - protein, 281 aa, unidentified
  REGION 1..281: note = Olavius algarvensis spirochete endosymbiont
MKFGAHAFVWEPEWNDTTSRRVISEAARIGLDFVEIPLLRPERFDGAATKVLLDEHAVGATYSLGLPSDKSLPERPYLAEPFLRSAIDAIESAGGDTLTGVLYGTLGELPGRPPNEKDYKVIAQVLRSVADYAKDRGIKLGIEPVNRYETFLVNTAEQAITLLDRIESDNVFIHLDTYHVNIEEDSFGAAIRLAGDRLGYIHLSESHRGTPGKGTVDWDDVFGALSDIGFAGPLVMESFVKLNADIARATCMWRDIVKDPEALIRDGIAFLEGKAKGYGLF

SEQ ID NO: 18 - DNA, 846 bp, unidentified
  misc_feature 1..846: note = Olavius algarvensis spirochete endosymbiont
  source 1..846: mol_type = genomic DNA
atgaaatttggtgcgcatgctttcgtctgggaaccagaatggaatgataccacctcgcgccgtgtgatttcagaggccgcgcgcatcggcctggattttgtggaaataccgctccttcgcccagaacgctttgacggcgccgctaccaaagtcttgctggatgaacatgccgtgggcgccacttattcattgggcctgcccagtgacaaaagcctgccagaacgcccgtatctggctgaacccttcttacggtctgcaatcgacgccattgaatccgcaggcggtgatacactgacaggcgtgctgtatggcactctgggtgagctgccgggccgcccgccgaacgaaaaggactacaaagtgattgcgcaggtattacgtagtgttgctgattacgcgaaagaccgtggcatcaaactgggcattgagccggtaaaccgttatgaaacctttctggtgaataccgcagagcaggcgatcacattgttagaccgtatcgaaagcgacaatgtctttattcatctggatacctatcacgtgaacatcgaagaagatagttttggcgcggctatccgcctggctggggatcgtttaggctacatccacctgtccgaaagccaccgtggcacgccaggcaagggtactgtggattgggacgacgttttcggagcgctttccgatattggtttcgcaggccctcttgtgatggaaagctttgtcaagctgaatgcagacattgcacgtgcgacctgcatgtggcgtgatattgtgaaagacccagaagccctgattcgcgatggtattgcgtttctcgaggggaaagcgaaaggctatggtctgttttaa

SEQ ID NO: 19 - protein, 282 aa, unidentified
  REGION 1..282: note = Acidiphilium
MKGFGIHTSLWAHDWTEQAARLAIPEAAKHGLAFVEIALIEPDRAETEVTRGLLEQHGLAACCSLGLPEEARPTTNPDKALEFVTLALEKTAAIGASLFTGVTYGSIGERTGQPPTAAELDAVARFLDKAAAVARGFGIIFGIEVVNRYESHLFNTTEQAVALIERVGAPNIVLHLDTYHMNIEATGQANAIRAAGAHLAYIHLSESHRGVPGTGTIAWDEVFAGLAGLGFTGGMALESFIHMPPRLAAALSVWRPVAPSRAAIIDEGLPFLRNKARQYGLI

SEQ ID NO: 20 - DNA, 849 bp, unidentified
  misc_feature 1..849: note = Acidiphilium
  source 1..849: mol_type = genomic DNA
atgaaaggctttggcattcatacaagtctgtgggcccatgactggaccgagcaagcggctcgtttagcaattccggaagcggcaaaacatggcctggcgtttgtcgaaattgctttaattgaaccagatcgcgcggaaactgaagttacccgcgggctgctggaacagcacggcttggctgcctgctgctctctgggcttacctgaggaagcgcggccaacgacgaacccggataaagcgttggagttcgtcactttagctctggaaaagaccgcggcgataggggctagcctgtttaccggtgtgacctacgggagcattggcgaacgtacgggccagccacctactgccgccgaacttgatgcagtagcaaggttcttggataaggctgccgcagtagcgcgcggtttcggtatcattttcggtatcgaagtagtgaatcggtacgagtctcatctgtttaacaccaccgaacaggccgttgcactgatcgaacgtgtcggcgcaccgaacatagtgctgcacttagacacgtatcacatgaacatcgaagccacgggccaggccaacgcaatacgcgcggcgggtgcacatctggcctatattcacctgagtgaatcgcacaggggtgttccgggcacagggaccatcgcctgggacgaggtgtttgcaggtcttgccggcctgggctttaccggcgggatggccctcgaatcatttattcacatgccgccccgcctggccgccgcgctgagcgtctggcgtccagttgcaccgagtcgcgcggccatcatcgatgaaggccttccatttctgcgaaataaagcccgtcaatacggcctcatttaa

SEQ ID NO: 21 - protein, 285 aa, unidentified
  REGION 1..285: note = Rhodobacterales bacterium
MLSLGLHALAAAPEWRPDLWAAILPRMAAHGVSVIEIPLLRPAELDIAGTRALAAKHDVELVCSLGLPATLNVAERPDEAFDFIRVALEVTASAGATALSGVTFGVIGQTTGAAPTTREIDAMTRHVSRSAALAKKLGLRFGIEPCNRYETHLLNTGAAARAVIERSGADNAFIHLDTYHMNIEEVSHAQGFADAGDLLGYVHLSESNRGVPGRGTVDWANVFQGLKAAGFDGCAALESFVFVDADLASGLAIWRPVAENPDDVIDVGFPFLRTAGEAAGLRWAR

SEQ ID NO: 22 - DNA, 858 bp, unidentified
  misc_feature 1..858: note = Rhodobacterales bacterium
  source 1..858: mol_type = genomic DNA
atgctttctctgggactgcatgcgcttgcagccgcccctgaatggcgcccggatctgtgggcggcgatcctgccgcgtatggcggcccacggtgttagcgttatcgaaattccgctgctccgtcctgctgaactggatattgcgggcacgcgggctttagcggctaaacatgatgttgaactcgtttgttcattggggctgccagccaccctcaatgttgcggaacgccctgatgaggcgtttgattttatccgcgtggcgttagaagtgaccgcgagcgcgggtgcgaccgcgctgtcaggagtgacatttggcgtgattgggcagacgacgggcgccgccccaaccacgcgcgaaatcgatgctatgacccggcatgtgagccgtagcgcagccctcgccaagaaactgggcttgcgctttggcattgagccgtgcaaccgctacgaaacccacttactgaatactggcgcagccgctcgggccgtgatagaacgctcaggcgctgataacgccttcatccatttggatacatatcacatgaatatcgaagaagtgtctcatgctcagggttttgcagatgcgggcgacctgctgggatatgtgcacttgtcagaaagcaatcgaggcgtcccggggcgtggtaccgtcgattgggcgaatgtatttcagggtctgaaagccgccggattcgatggttgcgccgcactcgaaagtttcgtgttcgttgacgcggacttagcatccggacttgccatctggcgtcctgttgctgaaaacccagatgacgtgattgacgtaggtttcccgttcttgcggacagccggagaggcggcgggcttgcgctgggcgcgttaa

SEQ ID NO: 23 - protein, 282 aa, unidentified
  REGION 1..282: note = Chloroflexi bacterium
MVLFGAHTFIWSAEWNPETAEHVIDGAARAGLDFVEIPLLHPDQMDAGGTRRLLENYGLQCTCSLGLPREAHLPFAPDKATGFLEQAVDVTSDLGSPVLTGCLYTHLGTLTGKPPTDEEIDLVVRVLKRIARYAQDRGISLGIEPVNRYETYLLNLAEQASALLDRIDEANVFVHLDTYHMNIEEKGFYTPIVDTGPRLQYIHLSESDRGIPGTGNVHWDEVFRGLKAVKYEGRLVMESFAAVNEDLMGATAMWRDVVGDPDRLVTEGLAFLRGKAIEYGLL

SEQ ID NO: 24 - DNA, 849 bp, unidentified
  misc_feature 1..849: note = Chloroflexi bacterium
  source 1..849: mol_type = genomic DNA
atggtactgtttggggcgcatacctttatttggagcgccgaatggaacccagaaacggcggaacatgtaattgatggcgcagctcgcgccggcctggatttcgttgagattccgttgctgcatcccgatcagatggacgcaggcggcacgcgccggcttttggaaaactatggtctgcaatgcacatgtagcttgggcctgccgcgcgaagcgcatctgccattcgcccctgacaaggcaacaggctttctggaacaggccgtcgatgtgacaagtgacctgggtagccctgttttgaccggctgtttatatacccacttaggaacgctgacaggaaagccgcctaccgacgaagagattgatttggtcgttcgtgtcctgaagcgaattgcgcgctacgcccaggaccgagggattagtctgggcatcgaaccggtcaaccgctatgaaacctatcttctgaatctggcggagcaagcgtctgcactgctcgatcgtattgacgaagccaatgtatttgtgcatctggatacctatcacatgaacattgaagaaaagggcttttatactccgatcgttgataccgggccgcgtttacagtacattcacctgtccgaatcggaccgcggtatcccgggtactggcaacgttcattgggacgaggtgtttcgaggcctcaaggccgttaaatacgaaggccgtcttgtgatggaatctttcgccgcagtcaacgaagatctgatgggtgcaacggcgatgtggcgggatgtggtgggtgatccggatcgattagtcacggaaggcctggcgttcttacgtggaaaggcgattgagtacggcctgctgtaa

SEQ ID NO: 25 - protein, 283 aa, unidentified
  REGION 1..283: note = Spiribacter sp. E85
MAEFGAHAFIWESDWNPASARRVIAGAAAAGLDFVEIPLLRPESMDTAGTRRLLAEHRLGVRCSLGLPPAASLPAHPQAAEAFLCRALDVTRALGGPVLTGVIYGTLGQLPGHPPRPGDLDIVAQTLRRVAAYAADQGLALGIEPVNRYETHLVNLTDQALELLDAIGADNVFLHLDTYHMNVEEKGFRGPVEAAGKRLRYIHLSESDRGTPGTGNVHWDEVFDGLAAIGYRGDLVMESFAAVNEDIARATCIWRQVAPDPDTLVREGLAFLRGKATARGLIP

SEQ ID NO: 26 - DNA, 852 bp, unidentified
  misc_feature 1..852: note = Spiribacter sp. E85
  source 1..852: mol_type = genomic DNA
atggcggagttcggtgcacatgctttcatttgggagtctgactggaatccggcgtcagctcgccgtgtcattgccggcgccgctgccgccggcctagatttcgtcgagatcccactgctccgcccggaatccatggacaccgcgggaacccgcagattacttgctgaacatcgcctcggcgtgcgctgttcactgggcctgccgccggcagcgagtctacccgctcatccgcaggccgcagaagcattcctttgccgcgccctagacgttacgcgtgcgttgggcggacccgtactcactggtgtcatctatggcactctgggccagttaccgggtcacccgccacgccctggcgatcttgacattgtcgcacagaccttacggcgcgtggcagcgtacgccgcagatcagggtctggccctgggcattgaaccagtgaaccgttatgaaacacatttagtgaatctcacggatcaagccctagaactgttagatgcgattggcgccgacaatgtattcctgcatctggatacctatcacatgaacgtggaagagaaaggttttcgtggtccagttgaagcagctggtaaacgtttgagatacattcatctttcggagtcagatcgtggtaccccggggacaggcaacgttcactgggatgaagtattcgacggtctggctgcaatcggataccgtggggatctggtgatggaaagttttgcggccgtgaatgaggacattgcgcgtgcgacctgcatctggcgtcaagttgccccagacccggacactctggttagagaaggcttggcgtttcttcgcggcaaagcgaccgcccgcggccttatcccttaa

SEQ ID NO: 27 - protein, 282 aa, unidentified
  REGION 1..282: note = Tabrizicola sp. TH137
MEGFGVHTSMWTMHWDRAGAERTIPAAAAYRMDFIEIALLNTAIVDAAHTRALLERHGMRAVCSLGLPERNWASVNPEGAIAHLCDCIDTAAAMGAEALSGVTYGGIGQRTGLPPTMAEYDNIARALAAVAKHAKARGIAFGIEPVNRYENHLINTAAQAKWMIEKVGADNIFIHLDTYHMNIEEKGAGNGILDARDHLRYIHLSESDRGTPGEGTCDWDEVYATLAAIGFKGGLAMESFINMPPEVAYGLAVWRPVAENFEEVMDKGLPFLRNKARQYRLI

SEQ ID NO: 28 - DNA, 849 bp, unidentified
  misc_feature 1..849: note = Tabrizicola sp. TH137
  source 1..849: mol_type = genomic DNA
atggaaggatttggtgtacatacttccatgtggacaatgcactgggatcgcgcgggcgctgaacgtaccattcctgccgcagctgcgtaccgcatggatttcatagagattgctttattaaacacagccattgttgacgcggcacacacgagggctctccttgaacggcatggcatgcgcgcagtgtgttcattgggcttacctgaacgcaattgggcgagcgtgaacccggaaggcgccatcgcacatctgtgcgattgtattgacacagcagcagccatgggcgccgaagccctatccggcgtcacgtatggcggcatcgggcaacgtacggggctcccgcctacgatggccgaatacgataacattgcccgcgctctggcagctgtggcgaaacatgctaaagcacgcggcattgcgtttggtatcgaacccgtgaatcgctacgaaaatcacttgattaataccgccgcacaagctaaatggatgatcgaaaaggttggggctgataatatatttattcatctggacacctatcacatgaacatagaggaaaagggagccggcaatgggatcctcgatgcgcgtgatcatttgagatatatccacttatcggaatcggatcgcggcactccgggcgaaggcacgtgtgactgggatgaggtatatgctaccctggccgccattggttttaaaggcggtttggctatggaatcgtttattaatatgccaccggaagtggcctatggtcttgccgtctggcgtccggtggcggaaaattttgaggaagtgatggataaaggactcccgttcttgcgtaataaagcgcgccaatatcgcctgatataa

SEQ ID NO: 29 - protein, 282 aa, unidentified
  REGION 1..282: note = Tropicimonas isoalkanivorans
MKGFGVHTSMWTMHWDRDGAERTIPATAAYGMDFVEIALLDTSIVDAAHTRALLEKHELRAVCSLGLPEGSWPSVAPEAAVAHLKDVFETAAAMGAEAVSGVTYGGIGQRSGVPPTEEEYDNVARALQQAAAYAKRLGLAFGIEPVNRYENHLINTAWQARDMIEKVGSDNIFIHLDTYHMNIEEKGVANGILDARDHLRYIHLSESDRGTPGEGCCDWNEVFGTLSAIGFEGGMAMESFINMPPQIAYGLAVWRPVADSFEDVMDRGLPFLRNKARQYRLA

SEQ ID NO: 30 - DNA, 849 bp, unidentified
  misc_feature 1..849: note = Tropicimonas isoalkanivorans
  source 1..849: mol_type = genomic DNA
atgaagggtttcggggtgcacacgagtatgtggacgatgcattgggaccgggatggcgcggaacgtacaattccagcgacggccgcgtatggcatggattttgtcgagatcgctctgttggatacctctatcgttgatgctgcgcacacccgggcgctcctggagaaacacgagctgcgcgccgtttgctcgttgggcctgccggaaggctcgtggccttcggttgccccagaggcggcggtagcgcatctgaaagacgtatttgaaaccgcagccgcgatgggcgccgaagcggtttcgggcgtcacatacggcggtattggccaacgttccggtgtgccgcccaccgaggaagaatatgacaacgtagctcgtgccctgcaacaggcagcggcttacgccaaacgcttaggcttagcctttggcattgaacctgtgaaccgctacgaaaaccatttgatcaacactgcatggcaggcccgtgacatgattgaaaaggtaggctcggacaatattttcatccatttagatacttaccacatgaatatagaagagaaaggtgtggcgaacggtatcttagacgcacgtgatcaccttcgttatattcatctttcagagagcgaccgtggcactccgggtgaaggttgctgcgattggaacgaagttttcgggaccttgagcgctattgggtttgaaggtggtatggcgatggaatcgtttattaacatgccgccgcagatagcttacggcctggccgtttggcgaccagttgcggactccttcgaagatgtgatggatcgcgggctgccgtttctgcgtaacaaagcgcgtcagtaccgtctggcttaa

SEQ ID NO: 31 - protein, 282 aa, unidentified
  REGION 1..282: note = Rhodobacteraceae bacterium
MQGFGVHTSMWTMHWDRLGAERTIPAAAAYKMDFIEIALLDTSVVDAHHTRDLLAKHEMRAVCSLGLPRESWASVNPDGAIAHLIDAMDYTKEMGGEALSGVTYGGIGERSGVPPTEAEYDNIARALEVAAKYAKTLGIAFGIEPVNRYESHLINTSWQAKEMIDKIGADNIFIHLDTYHMNIEEKGAGNGILAAREHLRYIHLSESDRGTPGEGTCDWDEIFATLAAVEFKGGLAMESFINMPPQVGYGLGIWRPVANSFEEVMDKGLPFLRNKAAQYRLI

SEQ ID NO: 32 - DNA, 849 bp, unidentified
  misc_feature 1..849: note = Rhodobacteraceae bacterium
  source 1..849: mol_type = genomic DNA
atgcaaggttttggtgtccataccagtatgtggacaatgcactgggatcgcctgggcgcggaacgaaccattccggccgcggctgcgtacaaaatggactttattgaaattgctcttctggacaccagcgtcgtggatgcgcatcacactcgtgatcttctggcgaaacatgaaatgcgtgcggtttgctcgctgggcttgcctcgcgaatcttgggccagtgttaacccggatggtgccatagctcaccttatagatgcaatggactatactaaagagatgggcggcgaggcactgtctggcgtcacctatggcggtatcggtgaacgtagcggtgttccgccgacggaagctgaatatgataatatcgcacgtgccctagaagttgccgccaaatacgccaaaacgctgggcattgcctttggcattgaacccgtgaaccgctatgaaagccacctgattaataccagttggcaggctaaagaaatgattgataaaatcggtgcagataacatcttcattcacttagatacctaccacatgaacattgaagagaaaggcgccggtaatggaatcctggcagctcgggaacaccttcgctacattcatttgagcgaaagcgaccggggtacacccggcgaaggcacctgtgattgggacgaaatatttgcgacccttgctgccgttgagttcaaaggtggcttggccatggagagtttcattaatatgccgccgcaagtgggatatggcttgggtatctggcgcccggtcgccaattcattcgaggaagtgatggataaaggcctgccgttcctgcgtaacaaagcggcccagtatcgtcttatttaa

SEQ ID NO: 33 - protein, 282 aa, unidentified
  REGION 1..282: note = Rhodobacter sp. SW2
MEGFGVHTSMWTMQWDRAGAERTIPAAAAYKMDFIEIALLNTAIVDAPHTRALLQKHGLRAVASLGLPQQNWASVNPEGAIEQLCRSLDTAAAMGCEALSGVTYGGIGERSGLPPTMAEYDNVARALAAAAKHAKKLGLAFGIEPVNRYESHLINTGWQAKWMIEKVGADNIFIHLDTYHMNIEEKGAGNGILDAREHLRYVHLSESDRGTPGEGTVDWDEIFATLAAVGFKGGLAMESFINMPPELGYGLAVWRPVAESFQAVMDKGLPFLRNKARQYRLI

SEQ ID NO: 34 - DNA, 849 bp, unidentified
  misc_feature 1..849: note = Rhodobacter sp. SW2
  source 1..849: mol_type = genomic DNA
atggaaggtttcggtgtccatacttccatgtggaccatgcaatgggaccgtgctggtgccgaacggacgataccagccgctgcggcatataagatggatttcatcgaaatcgcgctgctgaatacggccattgtggatgccccgcatacccgcgcactgttacagaaacatggtctgcgcgccgttgcgagtctgggcctgccacaacagaactgggcgagtgtcaatcctgagggtgccattgagcagttatgccgctcactggacaccgctgccgcgatgggttgcgaagccctgtcaggggttacctacggcggcatcggagagcgcagcggtctgccgccaacaatggcagaatatgacaacgtggcccgtgcgttggcggcggcagcaaaacacgctaagaaactgggcctggcgtttggaatcgaacctgtgaacagatatgagtctcacttgatcaacaccggatggcaagcaaaatggatgattgaaaaggtgggtgcggataacatctttatccacctggatacttatcacatgaatattgaggaaaagggtgcaggcaacggcattctcgatgcgcgcgaacacctgcgttacgtgcatcttagcgaaagcgaccgtgggacccctggagaaggtactgttgactgggatgaaatattcgcgacactcgccgcagttggatttaaaggcggattagcaatggaaagctttatcaacatgccaccggaactgggatatggcctggcagtatggcgcccggtggcggaatcctttcaagcagtaatggataaaggtttaccttttctgcggaacaaagcgcgtcagtatcgtctgatctaa

SEQ ID NO: 35 - protein, 282 aa, unidentified
  REGION 1..282: note = Alphaproteobacteria bacterium HGW-Alphaproteobacteria-8
MRGFGVHTSMWTMKWDRAGAERAVAAAAHYKMDFIEIALLNAPGVDAPHSRALLEKHGLRAVCSLGLPEAAWPSRNPEAAVAHLKVALDKTAEMGCEALTGVTYGGIGERTGLPPSATELDNVARALREAAAHAKKLGLLFGIEPVNRYETHLINTGRQAVEMIEKVGAENMFIHLDTYHMNIEEKGAANGILDAREHIKYIHLSESDRGTPGWGTCDWDEIFAVLSAIGFKGGLAMESFINMPPEVAYGLSVWRPVARDEAEVMDNGLPFLRGKARQYRLI

SEQ ID NO: 36 - DNA, 849 bp, unidentified
  misc_feature 1..849: note = Alphaproteobacteria bacterium HGW-Alphaproteobacteria-8
  source 1..849: mol_type = genomic DNA
atgcgtggttttggcgtccatacatctatgtggactatgaaatgggaccgtgcaggtgccgagcgtgccgtggcggcggcagcccactataagatggacttcatcgaaattgcgctgctgaacgcgccgggtgtcgatgcaccgcatagtcgggcactcctggagaaacatggactgcgcgcggtctgttcattggggcttccggaagccgcttggccgagccgcaacccggaagccgccgttgctcatctgaaagtggcgctcgataaaaccgctgaaatgggttgtgaagcgctgaccggagtcacctacggcggcatcggggaacggaccggcctgccaccgtctgccaccgaactggataacgtggcacgcgctttacgcgaagccgcagcacacgctaagaagctggggctcctcttcggcatcgaaccggttaaccgctacgaaacccatctgattaacactggtcggcaagccgttgaaatgattgagaaagtcggcgcagagaatatgtttattcacctggacacctatcacatgaacatcgaggaaaagggcgcagcgaatggtattctggatgcacgcgaacacattaaatatatccatctttcagaatctgatcgcggtaccccgggctggggtacctgcgattgggatgaaattttcgcagtgctgagcgcaattggatttaaaggtggtttggcgatggaaagttttattaacatgccgccggaagttgcgtatggcctgtcagtatggcgaccagtcgcacgggatgaagcggaagttatggataacggccttccgtttcttcgtggtaaagcccgccagtaccgcttaatttaa

SEQ ID NO: 37 - protein, 282 aa, unidentified
  REGION 1..282: note = Rubellimicrobium mesophilum
MQGFGVHTSMWTMNWDRAGAERTIPAASAYGMDFIEIALLNAPAVDAPHTRALLEKHGMRAVCSLGLPERNWASVNPEGAIEHLRQALEVTAALGAEALSGVTYGGIGQRTGVPPTAGEYDNIARALEAAARYARELGIAFGIEPVNRYENHLVNTAAQAKWMIEKVGADNIFIHLDTYHMNIEEKGVGNGILDAREHLRYIHLSESDRGTPGEGTCDWDEVFATLAAIGFKGGLAMESFINMPPEIGYGLAVWRPVAESFEEVMDRGLPFLRNKAKQYRLV

SEQ ID NO: 38 - DNA, 849 bp, unidentified
  misc_feature 1..849: note = Rubellimicrobium mesophilum
  source 1..849: mol_type = genomic DNA
atgcaagggtttggcgtccacacttcgatgtggaccatgaactgggatcgtgccggcgccgaacgtacaatcccggccgcgagcgcatacggtatggattttattgaaattgcgctgctgaatgccccggcggtggacgctccacatacgcgcgcccttcttgaaaagcacgggatgcgtgcagtttgcagtttgggtttgcctgagcgtaattgggcaagcgtcaaccctgaaggcgcgatcgagcaccttcgacaagccctcgaagtgaccgccgcccttggggcagaagcgttaagcggtgtcacatacggcggtataggtcagcgcacaggggtgccacccaccgctggcgagtatgataacatcgcccgtgcactggaagccgctgcacgctatgcacgagaactgggtatcgcctttggtatcgaaccggttaaccggtacgagaaccatctggtgaacaccgcggcccaagctaaatggatgatagaaaaggttggcgcggataatattttcatccatttggatacatatcacatgaacattgaggagaaaggtgtcggcaacggtatcctggatgcgcgtgagcacctgcgctatattcatctgtctgagagcgatcgcggcacaccaggcgagggtacttgcgattgggacgaagtattcgcaaccctggcagcaattggtttcaaaggcggacttgctatggaatcgtttatcaacatgccgccagaaattggatacggcttggccgtttggcgacctgtggcagagagttttgaagaagtcatggaccgcggcctgccgttcctgcggaacaaggcgaaacagtaccgtcttgtgtaa

SEQ ID NO: 39 - protein, 282 aa, unidentified
  REGION 1..282: note = Rhodobacterales bacterium
MEGFGVHTSMWTMHWDRAGAERTIPAAAAYKMDFIEIALLNAAMIDPPHTRALLEKHNMRAVASLGLPQRNWASVNPDGAAAHLIEAMDVAAAMGAEALSGVTYGGIGERTGLPPTMAEYDNIARALGQAAKHAKKLGIAFGIEPVNRYENHLINTGWQAKWMIEKVGADNIFIHLDTYHMNIEEKGAGNGILDAREHLRYIHLSESDRGTPGEGTCDWDEVYATLAAIGFKGGLAMESFINMPPEVGYGLAVWRPVANSFEEVMDKGLPFLRNKARQYRLI

SEQ ID NO: 40 - DNA, 849 bp, unidentified
  misc_feature 1..849: note = Rhodobacterales bacterium
  source 1..849: mol_type = genomic DNA
atggaaggcttcggtgtgcacacgagtatgtggacgatgcactgggatcgtgctggtgctgaacgtacgattccggccgccgccgcgtacaagatggacttcattgaaattgcgctgctgaacgcagcgatgatcgatccgccacatactcgcgcgcttctggagaaacataacatgcgggctgttgcgagtctcggacttccacaacgcaattgggcctcggttaatccagatggtgccgcggcccatcttatcgaggcgatggacgtggcggcagcaatgggtgccgaagcgctttccggagtgacatacggcggaatcggtgaacgcactggcctgccgccaactatggcagagtacgataacattgcacgagcactcgggcaggccgccaaacatgccaagaagcttgggatcgctttcggcatagagccggtgaatcgctatgagaatcatctgatcaacactggctggcaggccaaatggatgattgagaaagtgggtgctgataacatctttatccatctggatacatatcacatgaatatcgaggaaaagggcgctggtaatggtattttagatgcacgtgaacacctgcgttatattcatttgtctgagtccgatcgcggcacgccgggtgaaggcacgtgtgactgggatgaagtttacgcgacactggcagccattggctttaaaggtggactcgcgatggaatcattcatcaacatgccgccggaagtgggttatggtctggcagtttggcgcccggtggcgaacagctttgaagaagttatggacaaaggtctgccgtttctccgcaataaagcgcggcagtaccgcctgatctaa

SEQ ID NO: 41 - protein, 282 aa, unidentified
  REGION 1..282: note = Rhodobacterales bacterium RIFCSPHIGHO2_02_FULL_62_130
MEGFGVHTSMWTMKWDREGTERAVQAAVDYKMDFLEIALLDAPSVDAAHTRKLLEDNDMRAVCSLGLPEAVWPSRDPESAIAFMKGVFDKANEMGAEAVSGVTYGGIGERTDMPPTEAELSNVARALEACASYAKSLGLRFGIEPVNRYETHLLNTGWQARDMIERIGSDNIFIHLDTYHMNIEEKGAASGILDAREHLKYIHLSESDRGTPGEGCCDWDEIFATLAAINFTGGLAMESFINMPPELAHGLSVWRPVAPDFQAVMDKGLPFLRNKAAQYRLV

SEQ ID NO: 42 - DNA, 849 bp, unidentified
  misc_feature 1..849: note = Rhodobacterales bacterium RIFCSPHIGHO2_02_FULL_62_130
  source 1..849: mol_type = genomic DNA
atggaaggttttggggtacacaccagcatgtggacgatgaaatgggatcgtgagggcaccgagcgggctgtacaggcggcagttgattacaaaatggattttctggaaattgctctgcttgacgctccgagcgtggacgcggcacacacgcgcaaactcctggaagataacgatatgcgtgcggtgtgcagtttagggttaccagaagcggtgtggccgagtcgcgacccagagagcgcaattgcttttatgaaaggagtattcgataaggccaacgaaatgggcgccgaggcagtgagtggggtcacgtatggcggcataggcgaacgcacagacatgccaccgacagaagcggagctgagcaacgtagcccgcgcactggaagcgtgcgcgagttatgcaaaatcattaggtctgcgatttggcattgagcctgtgaatcgttatgaaacccatctgctgaataccggttggcaagcacgcgatatgatcgaacgtatcggttcagataacattttcattcatttggatacgtaccacatgaatatcgaagagaaaggcgcggcctctggtattttagatgcccgtgaacacttgaaatacatccatttatctgaaagtgacagaggcacgccaggcgaaggttgttgcgactgggatgaaattttcgccaccctcgccgccattaattttaccggcggtcttgccatggaaagtttcattaacatgccgccagagctggcgcatggtctgagcgtgtggcgccccgtggccccggacttccaagcggtgatggataaaggcctgccgttcctgcgaaacaaagccgcgcagtaccgcctggtctaa

SEQ ID NO: 43 - protein, 288 aa, unidentified
  REGION 1..288: note = Thermoclostridium caenicola
KYGIFYAYWEKEWKGDFITYIEKVKKLGFDILEVGCGDFHKQPDSYFHTLRDAAREYDIILTGGYGPRAEHNLCSPDTAVVENALAFYSDIFRKMEIAGIRSIGGGLYAYWPVDYSREPDKAGDLERSIKNMRRLADIAERHGITLNMEVLNRFEGYLINDTNEGLAYIRAVDKPNVKLMLDTFHMNIEEDSFTEPILQAGKYLGHVHVGEPNRKPPREGRIPWGEIGKALRQIGYDGPVVMEPFVTMGGQVGKDICVWRDLSQGATEEDLDRDAEKSLAFLKGMFEA

SEQ ID NO: 44 - DNA, 864 bp, unidentified
  misc_feature 1..864: note = Thermoclostridium caenicola
  source 1..864: mol_type = genomic DNA
aagtacggtatcttctatgcttattgggaaaaggaatggaagggtgacttcattacttacattgaaaaggttaagaagctgggattcgatattcttgaagttggttgtggtgacttccataaacaacctgattcttacttccatactcttagagatgctgctagagaatatgatattattcttactggtggttacggtcctagagctgaacataacttgtgttctccagatactgctgttgttgaaaacgctcttgccttctattctgatatcttcagaaaaatggagatcgctggtattagatccattggtggtggtctgtatgcttactggcctgttgattattctagagaacctgataaggctggtgatcttgaaagatccattaagaacatgagaagattggctgatattgctgaaagacatggtattactcttaatatggaagttcttaacagattcgaaggttatcttattaacgatactaacgaaggtcttgcttatattagagctgttgataaaccaaacgttaaattgatgttggataccttccacatgaacattgaagaagattccttcactgaacctattcttcaagctggtaagtatcttggtcatgttcatgttggtgaacctaatagaaagccacctagagaaggtagaatcccttggggtgaaattggtaaagctcttagacaaattggttacgatggtcctgttgttatggaaccattcgttactatgggtggtcaagttggtaaggatatctgtgtctggagagacttatctcaaggtgctactgaagaggatcttgatagagatgctgaaaagtctcttgccttcttgaaaggtatgttcgaagct

(19) United States
(12) Patent Application Publication
(10) Pub. No.: US 2023/0313254 A1
(43) Pub. Date: Oct. 5, 2023

(54) D-ALLULOSE 3-EPIMERASES FOR BIOCONVERSION OF D-FRUCTOSE TO D-ALLULOSE

(71) Applicant: Conagen Inc., Bedford, MA (US)

(72) Inventors: Guohong Mao, Burlington, MA (US); Michael Batten, Westford, MA (US); Oliver Yu, Lexington, MA (US); Timothy Joseph Hanly, Waltham, MA (US); Phillip James Hunt, Tewksbury, MA (US); Hongfang Liu, Wuxi (CN); Yang Luo, Wuxi (CN)

(73) Assignee: Conagen Inc., Bedford, MA (US)

(21) Appl. No.: 18/054,195

(22) Filed: Nov. 10, 2022

Related U.S. Application Data
(63) Continuation of application No. PCT/US21/31859, filed on May 11, 2021.
(60) Provisional application No. 63/022,617, filed on May 11, 2020.

(51) Int. Cl. C12P 19/24; C12N 9/90; C12N 15/70; C12N 15/81

(52) U.S. Cl. CPC C12P 19/24; C12N 9/90; C12N 15/70; C12N 15/81; C12Y 501/03; C12R 2001/19

(57) ABSTRACT

Provided herein are methods for identifying and isolating polynucleotides coding for polypeptides having D-allulose 3-epimerase activity from a wide variety of microorganisms. Also provided are nucleic acid constructs, vectors and recombinant host cells comprising the polynucleotides coding for D-allulose 3-epimerase activity as well as methods for producing allulose from fructose using said recombinant host cells having D-allulose 3-epimerase activity or the D-allulose 3-epimerase enzyme of said recombinant host cells having D-allulose 3-epimerase activity.

### RELATED APPLICATION

This application claims the benefit under 35 U.S.C. § 119(e) of U.S. Provisional Application No. 63/022,617, filed May 11, 2020, entitled “D-ALLULOSE 3-EPIMERASES FOR BIOCONVERSION OF D-FRUCTOSE TO D-ALLULOSE,” the contents of which are hereby incorporated by reference in their entirety.

### FIELD OF INVENTION

The present invention relates to a method of producing D-allulose from fructose using the D-allulose 3-epimerase enzymes. This invention provides polypeptides having D-allulose 3-epimerase activity, and nucleic acid molecules encoding said polypeptides. The invention also relates to recombinant nucleic acid constructs including vectors and recombinant host cells comprising the recombinant nucleic acid constructs.

### BACKGROUND

D-allulose, also known as D-psicose, is a type of sugar that structurally resembles fructose, which is the sugar that occurs naturally in fruits. D-allulose is available in a granulated form and looks like sucrose crystals. Allulose is a low calorie sweetener that has 70% of the sweetness of sucrose. According to the United States Food and Drug Administration (FDA), allulose provides about 0.4 calories per gram, which is significantly lower than the 4 calories per gram provided by sucrose. Although the human body has the capacity to absorb allulose, it lacks the capacity to metabolize allulose. As a result, allulose has little to no effect on blood glucose or insulin levels. For this reason, allulose is considered as a low-calorie sweetener.

Allulose is found naturally in some foods, such as dried fruits, brown sugar, and maple syrup in extremely small quantities. Chemical synthesis of allulose is difficult and there is a need in the field to produce allulose from readily available sugars such as fructose using recombinant microorganisms.

### SUMMARY

Enzymes from the ketose 3-epimerase family including D-allulose 3-epimerase (DAE, also named as D-psicose 3-epimerase (DPE)) can catalyze the reversible conversion of D-fructose into D-allulose. D-allulose 3-epimerases can have high activities for the bioconversion of D-fructose to D-allulose. The present invention provides, among other things, a method for producing D-allulose from D-fructose using recombinant polypeptides having D-allulose 3-epimerase activity.

In one embodiment, the present method involves contacting a fructose substrate with a reaction mixture that includes an enzyme system comprising a recombinant polypeptide having D-allulose 3-epimerase enzyme activity under conditions such that the D-fructose substrate is converted into D-allulose. In one aspect of this invention, the enzyme system provides a recombinant polypeptide having D-allulose 3-epimerase enzyme activity and D-fructose as a substrate to produce D-allulose as a product. In yet another aspect of the present invention, the present method can involve contacting a fructose substrate with a reaction mixture that includes a host cell capable of expressing an exogenous D-allulose 3-epimerase or a lysate of such a host cell as the source of the D-allulose 3-epimerase enzyme. In certain aspect of this embodiment, the D-allulose 3-epimerase enzyme present in the enzyme system is a purified enzyme and it can be derived from the lysate of a host cell expressing a D-allulose 3-epimerase enzyme using one or more biochemical techniques known in the art. In yet another aspect of the present invention, the purified form of D-allulose 3-epimerase is immobilized on a solid support. In various embodiments, the present method can further include adding at least one type of metal ions to the reaction system. For example, the metal ions can be selected from the group consisting of magnesium ions, manganese ions, copper ions, zinc ions, nickel ions, cobalt ions, iron ions, aluminum ions, and calcium ions. In some preferred embodiments, manganese ions and/or magnesium ions are added to the reaction system. The metal ions can be added at a concentration from about 0.01 mM to about 5 mM (e.g., 0.01 mM, 0.05 mM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, or 5 mM). In various aspects, the present method can include removing a product stream comprising allulose free from the remaining fructose in the reaction system.

In another embodiment, the present invention provides polynucleotide sequences coding for polypeptides having D-allulose 3-epimerase activity. In one aspect of this embodiment, the polynucleotide coding for polypeptide having D-allulose 3-epimerase activity is cloned into an appropriate plasmid expression vector having required regulatory elements such as promoter and terminator. In one aspect, the plasmid expression vector has an inducible promoter so that the expression of the D-allulose 3-epimerase enzyme activity can be induced by using certain chemicals.

In yet another embodiment, the expression plasmid vector comprising a polynucleotide sequence coding for a polypeptide having D-allulose 3-epimerase activity is used to transform a host cell including a prokaryotic microbial cell or a eukaryotic microbial cell or a eukaryotic animal cell or a eukaryotic plant cell. In one aspect, the expression plasmid vector within the transformed host cell exists as a self-replicating nucleic acid entity. In another aspect of this embodiment, the expression plasmid vector is integrated into the host chromosomal DNA.

In another embodiment, the present invention provides a method for recovering D-allulose free from D-fructose as a substrate in the reaction involving D-allulose 3-epimerase.

In some embodiments, the method of producing allulose described herein comprises contacting a fructose substrate with a reaction mixture comprising:

(a) an enzyme system comprising a D-allulose 3-epimerase enzyme;

(b) a host cell transformed with a recombinant vector comprising a nucleic acid molecule that encodes a D-allulose 3-epimerase enzyme; and/or (c) a lysate of the host cell of (b), under conditions such that the fructose substrate is converted into allulose, wherein the D-allulose 3-epimerase enzyme comprises an amino acid sequence having at least 80% (e.g., at least 80%, at least 85%, at least 90%, at least 95%, at least 98%, at least 99%, or 100%) sequence identity to a polypeptide selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43. In some embodiments, the reaction mixture comprises a D-allulose 3-epimerase enzyme comprising an amino acid sequence having at least 80% (e.g., at least 80%, at least 85%, at least 90%, at least 95%, at least 98%, at least 99%, or 100%) sequence identity to a polypeptide selected from the group consisting of SEQ ID NO:1, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, and SEQ ID NO:25.

In some embodiments, the D-allulose 3-epimerase enzyme comprises an amino acid sequence having at least 90% (e.g., 90%, 91%, 92%, 93%, 94%, 95%, 96%, 97%, 98%, 00%, or 100%) sequence identity to a polypeptide selected from the group consisting of SEQ ID NO:1, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, and SEQ ID NO:25.

In some embodiments, the D-allulose 3-epimerase enzyme comprises an amino acid sequence having at least 95% (e.g., 95%, 96%, 97%, 98%, 00%, or 100%) sequence identity to a polypeptide selected from the group consisting of SEQ ID NO:1, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, and SEQ ID NO:25.

In some embodiments, the D-allulose 3-epimerase enzyme comprises the amino acid sequence of SEQ ID NO:1, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, or SEQ ID NO:25. In some embodiments, the D-allulose 3-epimerase enzyme comprises the amino acid sequence of SEQ ID NO: 5.

In some embodiments, the reaction mixture comprises a D-allulose 3-epimerase enzyme comprising an amino acid sequence having at least 80% (e.g., at least 80%, at least 85%, at least 90%, at least 95%, at least 98%, at least 99%, or 100%) sequence identity to a polypeptide selected from the group consisting of SEQ ID NO:3, SEQ ID NO:11, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43.

In some embodiments, the D-allulose 3-epimerase enzyme comprises an amino acid sequence having at least 90% (e.g., 90%, 91%, 92%, 93%, 94%, 95%, 96%, 97%, 98%, 00%, or 100%) sequence identity to a polypeptide selected from the group consisting of SEQ ID NO:3, SEQ ID NO:11, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43.

In some embodiments, the D-allulose 3-epimerase enzyme comprises an amino acid sequence having at least 95% (e.g., 95%, 96%, 97%, 98%, 00%, or 100%) sequence identity to a polypeptide selected from the group consisting of SEQ ID NO:3, SEQ ID NO:11, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43.

In some embodiments, the D-allulose 3-epimerase enzyme comprises the amino acid sequence of SEQ ID NO:3, SEQ ID NO:11, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43.

In some embodiments, the conditions comprise maintaining the enzyme system and the fructose substrate at a temperature between 25° C. and 75° C. (e.g., 25° C., 30° C., 35° C., 40° C., 45° C., 50° C., 55° C., 60° C., 65° C., 70° C., or 75° C.).

In some embodiments, the conditions comprise maintaining the enzyme system and the fructose substrate at a pH between 4 and 10 (e.g., 4, 4.5, 5, 5.5, 6, 6.5, 7, 7.5, 8, 8.5, 9, 9.5, or 10).

In some embodiments, the D-allulose 3-epimerase enzyme is in isolated form. In some embodiments, the D-allulose 3-epimerase enzyme is immobilized on a solid substrate.

In some embodiments, the reaction mixture comprises a host cell transformed with a recombinant vector comprising a nucleic acid molecule that encodes the D-allulose 3-epimerase enzyme or a lysate of said host cell, wherein the nucleic acid molecule encoding the D-allulose 3-epimerase enzyme comprises a polynucleotide sequence having at least 80% (e.g., at least 80%, at least 85%, at least 90%, at least 95%, at least 98%, at least 99%, or 100%) sequence identity to a polynucleotide sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40,SEQ ID NO:42, and SEQ ID NO: 44. In some embodiments, the nucleic acid molecule encoding the D-allulose 3-epimerase enzyme comprises the polynucleotide sequence of SEQ ID NO: 44.

In some embodiments, the host cell is selected from the group consisting of a yeast cell, a filamentous fungal cell, a bacterial cell, a mammalian cell, a plant cell, and a Labryinthulomycetes cell. In some embodiments, the host cell is E. coli or P. pastoris. In some embodiments, the recombinant vector exists as a self-replicating nucleic acid molecule within the host cell. In some embodiments, the recombinant vector is integrated into the host cell chromosome.

In some embodiments, the method further comprises adding at least one type of metal ions to the reaction system, said at least one type of metal ions selected from the group consisting of magnesium ions, manganese ions, copper ions, zinc ions, nickel ions, cobalt ions, iron ions, aluminum ions, and calcium ions. In some embodiments, the metal ions are added at a concentration from about 0.01 mM to about 5mM (e.g., 0.01 mM, 0.05 mM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, or 5 mM). In some embodiments, the method further comprises removing a product stream comprising allulose from the reaction system as the fructose substrate is converted into allulose.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1. Bioconversion of D-fructose to D-allulose by D-allulose 3-epimerase (DAE). FIGS. 2A-2B. Enzymatic screening of D-allulose 3-epimerase candidates. The purified candidate recombinant polypeptides with D-allulose 3-epimerase activity were dialyzed against 50mM phosphate buffer (pH 7.2) and assayed for D-allulose synthesis by using D-fructose as substrate. Typically, the recombinant polypeptide (5-10 μg) was tested in a 200 μl in vitro reaction system. The reaction system contained 20 mM Tris-HCl buffer, pH 8.0, 3mM MgCl₂ or MnSO4, 20 g/L D-fructose. The reaction was performed at 60° C. and reaction was terminated at 2 hour (FIG. 2A) and at 16 hour (FIG. 2B) by heating for 10min. The samples were analyzed by HPLC for determining the amount of D-fructose and D-allulose.

FIG. 3. Map of the pHKA-AL39 plasmid construct. FIGS. 4A-4B. Identification and characterization of the AL enzyme produced by an engineered Pichia pastoris strain. FIG. 4A depicts an SDS-PAGE (16%) analysis of secreted AL39 enzyme from the culture medium of an engineered Pichia pastoris strain. The AL39 enzyme is indicated by an arrow. M: Protein ladder; S: Sample (12μL) of the culture medium of the engineered Pichia pastoris strain. FIG. 4B depicts DAE activity. Culture media were analyzed for the presence of fructose and allulose following a 1 hour reaction.

FIG. 5. Comparison of DAE activity of the immobilized AL39 enzyme across reuse cycles (1-5).

### DETAILED DESCRIPTION

Methods for producing allulose according to the present invention comprises contacting a fructose substrate with a reaction mixture that includes a polypeptide having D-allulose 3-epimerase activity. D-fructose is the preferred substrate and D-allulose 3-epimerase is the preferred enzyme. The reaction mixture according to the present invention can further comprise one or more cofactors. A divalent cation is a preferred cofactor in the reaction mixture according to the present invention. Manganese and magnesium are preferred divalent cations and they can be supplied as MgCl₂ and MnSO₄ respectively. Other suitable divalent cations can include copper ions, zinc ions, nickel ions, cobalt ions, iron ions, aluminum ions, and calcium ions. In one embodiment of the present invention, D-allulose 3-epimerase used according to the present invention is provided as a highly purified homogenous recombinant protein in a suitable buffer. In another embodiment of the present invention, D-allulose 3-epimerase used according to the present invention is provided as a highly purified recombinant protein immobilized on a solid support. In another embodiment of the present invention, the reaction mixture includes a recombinant prokaryotic cell or a recombinant eukaryotic cell expressing a recombinant D-allulose 3-epimerase and that recombinant prokaryotic cell or a recombinant eukaryotic cell is used as a source of D-allulose 3-epimerase.

A host cell according to the present invention is any cell that is suitable for the expression of a heterologous protein having D-allulose 3-epimerase activity. Such a host cell expressing heterologous protein having 3-epimerase activity results from the transformation of the host cell with a recombinant plasmid comprising polynucleotide sequence coding for a polypeptide having D-allulose 3-epimerase activity and such a host cell is referred as a recombinant host cell in the present invention. The list of host cells suitable for the present invention includes, but is not limited to, bacterial cells, yeast cells, plant cells, animal cells, and Labyrinthulomycetes cells. In some embodiments, the cellular system comprises bacterial cells, yeast cells, or a combination thereof. Bacterial cells suitable for the present invention include, without limitation, Escherichia spp., Streptomyces spp., Zymomonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacteriumspp., Pantoea spp, and Vibrio natriegens. Yeast cells suitable for the present invention include, without limitation, engineered Saccharomyces spp., Schizosaccharomyces, Hansenula, Candida, Kluyveromyces, Yarrowia, Candida boidinii, and Pichia.

The term a cell culture refers to any cell or cells including the recombinant host cells that are in a culture. Culturing is the process in which cells are grown under controlled conditions, typically outside of their natural environment. For example, cells, such as yeast cells, may be grown as a cell suspension in liquid nutrient broth. A cell culture includes, but is not limited to, a bacterial cell culture, a yeast cell culture, a plant cell culture, and an animal cell culture.

In some embodiments, cells are cultured at a temperature of 16° C. to 40° C. For example, cells may be cultured at a temperature of 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. or 40° C.

In some embodiments, cells are cultured for a period of 12 hours to 72 hours, or more. For example, cells may be cultured for a period of 12, 18, 24, 30, 36, 42, 48, 54, 60, 66, or 72 hours. Typically, cells, such as bacterial cells, are cultured for a period of 12 to 24 hours. In some embodiments, cells are cultured for 12 to 24 hours at a temperature of 37° C. In some embodiments, cells are cultured for 12 to 24 hours at a temperature of 16° C.

In some embodiments, cells are cultured to a density of 1×108 (OD₆₀₀<1) to 2×10¹¹ (OD˜200) viable cells/ml cell culture medium. In some embodiments, cells are cultured to a density of 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, or 2×10¹¹ viable cells/ml. (Conversion factor: OD 1=8×108 cells/ml).

To induce protein expression by the host cell, 0.5 mM isopropyl β-D-1-thiogalactopyranoside (IPTG) was added and the culture was further grown at 16° C. for 22 hr. Cells were harvested by centrifugation (3,000×g; 10 min; 4° C.). The cell pellets were collected and were either used immediately or stored at −80° C.

In some embodiments, cell pellets are harvested from the host cells expressing polypeptide having D-allulose 3-epimerase activity. In some embodiments, the host cell pellets may be resuspended at various concentrations. In some embodiments, the host cell pellets are resuspended at a concentration of 1 g/L to 250 g/L. In some embodiments, the host ell pellets harvested from the cellular system are resuspended at a concentration of 1 g/L, 10 g/L, 25 g/L, 50 g/L, 75 g/L, 100 g/L, 125 g/L, 150 g/L, 175 g/L, 200 g/L, 225 g/L, or 250 g/L.

The terms “incubating” and “incubation” as used herein refers to a process of mixing two or more chemical or biological entities or at least one chemical entity and at least one biological entity (such as a chemical compound and an enzyme) and allowing them to interact under conditions favorable for producing a product such as D-allulose as described herein.

The term “down-stream separation process” means recovering the ed product D-allulose from the original substrate I)-fructose. When fructose is used as a substrate in the enzymatic reaction to produce D-allulose, fructose is not fully consumed in the reaction and at the end of the enzymatic reaction a significant amount of fructose is still present in the reaction medium. D-fructose and D-allulose have similar physical and chemical properties and it is difficult to sperate them at the end of enzymatic reaction involving D-allulose 3-epimerase and fructose as the substrate. One possible way to recover D-allulose free from D-allulose is to is to convert the remaining fructose into mannitol and separating the D-allulose from mannitol. The NADPH- or NADH-dependent mannitol dehydrogenase can be used to convert D-fructose into mannitol. Mannitol dehydrogenase can be used along with formate dehydrogenase in a two-enzyme system to regenerate the reduced cofactors NADH required for the action of mannitol dehydrogenase. Formate dehydrogenase converts formate to carbon dioxide and reduces NAD to NADH. Mannitol can be crystallized from the aqueous solution through cold crystallization and D-allulose can be recovered in a form free from D-fructose, (Saha, B. C. and Racine, F. M. (2011) Biotechnological production of mannitol and its applications. Appl Microbiol Biotechnol. 89:879-891; U.S. Pat. No. 10,266,862B2).

The term immobilization refers to binding the host cell expressing D-allulose 3-epimerase or a purified polypeptide having D-allulose 3-epimerase activity to solid support using one or other methods well-known in the art. Sodium alginate, derived for marine algae can be used a solid support to immobilize the host cell expressing D-allulose 3-epimerase or a purified polypeptide having D-allulose 3-epimerase activity as described in detail in the patent document US2019169591A1.

The terms “nucleic acid” and “nucleotide” are used according to their respective ordinary and customary meanings as understood by a person of ordinary skill in the art, and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The terms “polypeptide,” “protein,” and “peptide” are used according to their respective ordinary and customary meanings as understood by a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polypeptide product. Thus, exemplary polypeptides include polypeptide products, naturally occurring proteins, homologs, orthologs, paralogs, fragments and other equivalents, variants, and analogs of the foregoing.

The term “functional fragment” of a polypeptide or protein refers to a peptide fragment that is a portion of the full length polypeptide or protein, and has substantially the same biological activity, or carries out substantially the same function as the full length polypeptide or protein (e.g., carrying out the same enzymatic reaction).

The term “functional variant” further includes conservatively substituted variants. The term “conservatively substituted variant” refers to a peptide having an amino acid sequence that differs from a reference peptide by one or more conservative amino acid substitutions, and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., Cell 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical.

“Percent (%) amino acid sequence identity” with respect to the variant polypeptide sequences of the subject technology refers to the percentage of amino acid residues in a candidate sequence that are identical with the amino acid residues of a reference polypeptide, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, the % amino acid sequence identity may be determined using the sequence comparison program NCBI-BLAST2. The NCBI-BLAST2 sequence comparison program may be downloaded from ncbi.nlm.nih.gov. NCBI BLAST2 uses several search parameters, wherein all of those search parameters are set to default values including, for example, unmask yes, strand=all, expected occurrences 10, minimum low complexity length=15/5, multi-pass e-value=0.01, constant for multi-pass=25, drop off for final gapped alignment=25 and scoring matrix=BLOSUM62. In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Techniques for determining amino acid sequence “similarity” are well known in the art. In general, “similarity” refers to the exact amino acid to amino acid comparison of two or more polypeptides at the appropriate place, where amino acids are identical or possess similar chemical and/or physical properties such as charge or hydrophobicity. A “percent similarity” may then be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded therein, and comparing this to a second amino acid sequence. In general, “identity” refers to an exact nucleotide to nucleotide or amino acid to amino acid correspondence of two polynucleotides or polypeptide sequences, respectively. Two or more polynucleotide sequences can be compared by determining their “percent identity”, as can two or more amino acid sequences. The programs available in the Wisconsin Sequence Analysis Package, Version 8 (available from Genetics Computer Group, Madison, Wis.), for example, the GAP program, are capable of calculating both the identity between two polynucleotides and the identity and similarity between two polypeptide sequences, respectively. Other programs for calculating identity or similarity between sequences are known by those skilled in the art.

An amino acid position “corresponding to” a reference position refers to a position that aligns with a reference sequence, as identified by aligning the amino acid sequences. Such alignments can be done by hand or by using well-known sequence alignment programs such as ClustalW2, Blast 2, etc.

Unless specified otherwise, the percent identity of two polypeptide or polynucleotide sequences refers to the percentage of identical amino acid residues or nucleotides across the entire length of the shorter of the two sequences.

The term “expression” as used herein, is used according to its ordinary and customary meaning as understood by a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is used according to its ordinary and customary meaning as understood by a person of ordinary skill in the art, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal DNA. Host organisms containing the transformed nucleic acid fragments are referred to as “transgenic” or “recombinant” or “transformed” organisms.

The terms “transformed,” “transgenic,” and “recombinant,” when used herein in connection with host cells, are used according to their ordinary and customary meanings as understood by a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are used according to their ordinary and customary meanings as understood by a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are used according to their ordinary and customary meanings as understood by a person of ordinary skill in the art, and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Standard recombinant DNA and molecular cloning techniques used herein are well known in the art and are described, for example, by Sambrook, J., Fritsch, E. F. and Maniatis, T. Molecular Cloning: A Laboratory Manual, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-Interscience, 1987; the entireties of each of which are hereby incorporated herein by reference to the extent they are consistent herewith.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### EXAMPLES

### Example 1: Construction of Plasmid Vectors for Expressing D-allulose 3-Epimerase

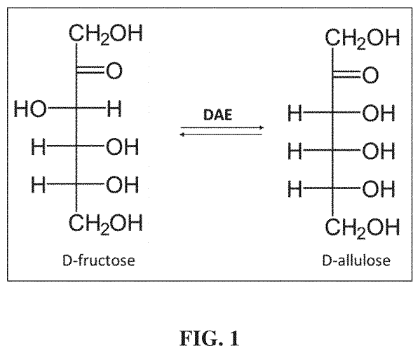

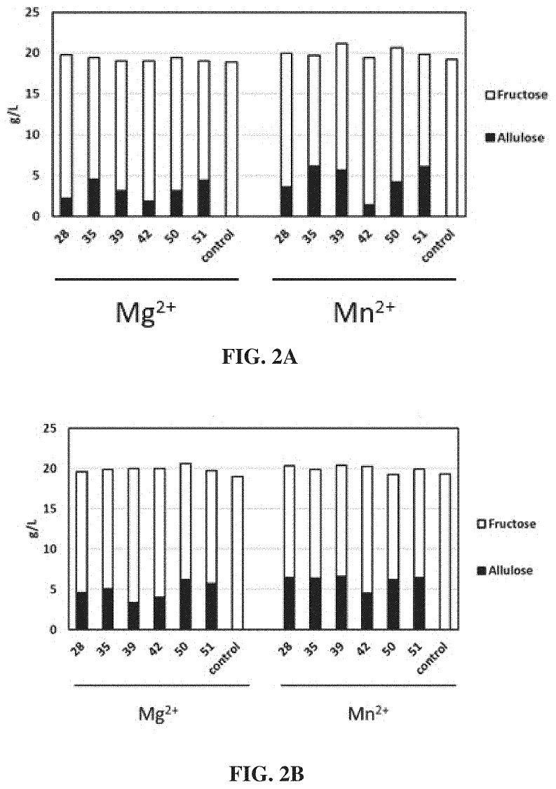

D-allulose 3-epimerase (DAE) has the ability to convert D-fructose to D-allulose (FIG. 1). In order to identify specific D-allulose 3-epimerase enzyme for D-allulose production using D-fructose as a substrate, the polynucleotide sequences coding for polypeptides having D-allulose 3-epimerase activity were identified using polygenetic and BLAST analysis. Full length DNA fragments of all candidate D-allulose 3-epimerase genes were commercially synthesized. Almost all codons of the cDNA were changed to those preferred for E. coli (Twist Bioscience, CA). The synthesized DNA was cloned into a bacterial expression vector to generate the expression construct.

### Example 2: Measurement of D-allulose 3-Epimerase Enzyme Activity in Recombinant E. coli Cells

Each expression vector construct with the polynucleotide coding for a polypeptide having D-allulose 3-epimerase activity prepared as in Example 1 was transformed into E. coli T7 Express cell (Biolabs, MA), which was subsequently grown in Terrific Broth media containing 50 μg/mL ampicillin at 37° C. until reaching an OD600 of 0.8-1.0. Protein expression was induced by addition of 0.5 mM isopropyl β-D-1-thiogalactopyranoside (IPTG) and the culture was further grown at 16° C. for 22 hr. Cells were harvested by centrifugation (3,000×g; 10 min; 4° C.). The cell pellets were collected and were either used immediately or stored at −80° C.

The cell pellets were extracted by extraction buffer (BugBuster Master Mix, EMD Millipore, US). After centrifugation, the supernatant was added in the reaction mixture for activity assay. Typically, the supernatant (5 μl) was tested in a 200 μl in vitro reaction system. The reaction system contains 20 mM Tris-HCl buffer, pH 8.0, 1 mM MgCl₂ or MnSO4, 10 g/L D-fructose. The reaction was performed at 60° C. and reaction was terminated by heating for 10 min. The samples were analyzed by HPLC.

The concentration of D-fructose and D-allulose were determined by an HPLC system (Vanquish, Thermo Scientific, USA) equipped with RefractorMax521 detector (IDEX Health & Science KK, Japan). The chromatographic separation was performed using Rezex RCM-Monosaccharide Ca²⁺ column (100×7.8 mm, Phenomenex, CA). The column was eluted at 80° C. with water at a flow rate of 0.5 ml/min. From the analysis of D-allulose 3-epimerase enzyme activity, a total of 21 candidate D-allulose 3-epimeras enzyme having appropriate enzymatic activity for bioconversion of D-fructose to D-allulose were identified (Table 1).

### Example 3: Purification of Recombinant D-allulose 3-Epimerase

In order to purify the recombinant protein, the cell pellets typically were re-suspended in lysis buffer (50 mM potassium phosphate buffer, pH 7.2, 25 mg/ml lysozyme, 5 mg/ml DNase I, 20 mM imidazole, 500 mM NaCl, 10% glycerol, and 0.4% Triton X-100). The cells were disrupted by sonication under 4° C., and the cell debris was clarified by centrifugation (18,000×g; 30 min). Supernatant was loaded to an affinity column Ni-NTA (Qiagen) equilibrated with an equilibration buffer containing 50 mM potassium phosphate buffer, pH 7.2, 20 mM imidazole, 500 mM NaCl and10% glycerol. After loading of protein sample, the column was washed with equilibration buffer to remove unbound contaminant proteins. The His-tagged recombinant polypeptides with D-allulose 3-epimerase activity were eluted by equilibration buffer containing 250 mM imidazole.

The purified candidate recombinant polypeptides with D-allulose 3-epimerase activity were dialyzed against 50 mM phosphate buffer (pH 7.2) and assayed for D-allulose synthesis by using D-fructose as substrate. Typically, the recombinant polypeptide (5-10 μg) was tested in a 200 μl in vitro reaction system. The reaction system contained 20 mM Tris-HCl buffer, pH 8.0, 3 mM MgCl₂ or MnSO4, 20 g/L D-fructose. The reaction was performed at 60° C. and reaction was terminated at 2 hour and at 16 hour by heating for 10 min. The samples were analyzed by HPLC.

These candidate recombinant polypeptides with D-allulose 3-epimerase activity have various enzymatic activity for allulose production with different preference for divalent metal factor. As shown in FIGS. 2A-2B, all selected candidates have D-allulose 3-epimerase activity at 2 hr (FIG. 2A) and more D-allulose can be produced when the reactions were extended to 16 hr (FIG. 2B). Compared to Mg²⁺ addition, Mn²⁺ addition can increase the activity of majority enzymes (AL28, 35, 39, 42, 50 and 51), indicating these enzymes prefer to Mn²⁺ cofactor.

### Example 4: Generation of an Engineered Pichia pastoris Strain to Produce DAE Enzyme

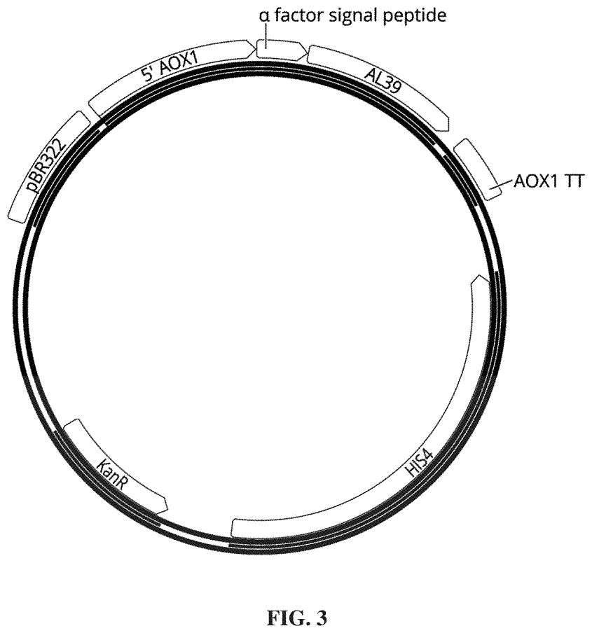

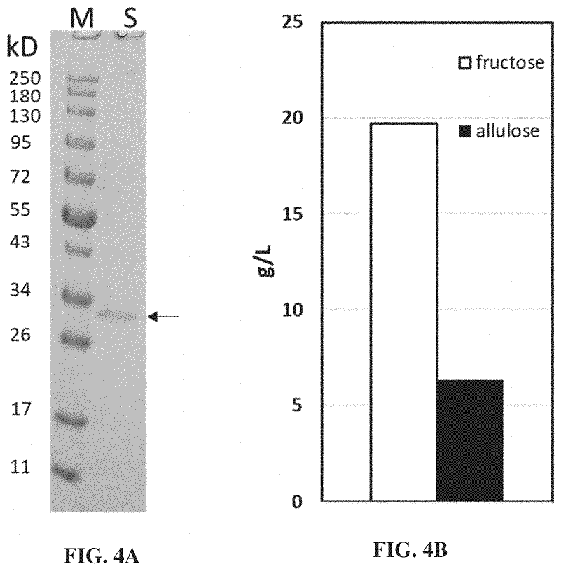

Full-length DNA fragments of the AL39 gene (SEQ ID NO: 43) were synthesized for use in the transformation of the Pichia pastoris cells. Specifically, the cDNA was codon optimized for Pichia pastoris expression to produce the AL39 enzyme (SEQ ID NO: 5). The AL39 gene was inserted in frame with a-mating factor signal peptide. The synthesized fusion gene was cloned into a pHKA vector (a modified Pichia pastoris expression vector), using EcoRI and NotI restriction digestion. In the expression plasmid (pHKA-AL39, FIG. 3), the expression cassette contains an AOX1 promoter, an α-mating factor signal peptide, the AL39 gene and an AOX1 transcription terminator.

The linearized pHKA-AL39 plasmid was transformed into Pichia pastoris (GS115) cells using methods known in the art (Lin-Cereghino, et al., Biotechniques, 38(1):44-48, 2005) and the expression cassette was integrated into the Pichia pastoris genome at the HIS4 locus.

To demonstrate the secreted AL39 enzyme production, a single colony of the Pichia pastoris strain pHKA-AL39 was inoculated in BMGY medium in a baffled flask and grown at 28-30° C. in a shaking incubator (250-300 rpm) until the culture reached an OD600 of 2-6 (log-phase growth). The pHKA-AL39 cells were harvested by centrifuging and resuspended to an OD600 of 1.0 in BMMY medium to induce expression. 100% methanol was added to the BMMY medium to a final concentration of 1% methanol every 24 hours to maintain induction of expression. The media from pHKA-AL39 culture was harvested by centrifugation and subjected to DAE activity analysis and SDS-PAGE analysis as described below.

After centrifugation, the supernatant was added in the reaction mixture for activity assay. Typically, the supernatant (650) was tested in a 200 μl in vitro reaction system. The reaction system contained 20 mM Tris-HC buffer, pH 8.0, 1 mM MnSO4, 30 g/L D-fructose. The reaction was performed at 55-60° C. and terminated by heating for 10 min. The samples were analyzed by HPLC.

The concentration of D-fructose and D-allulose were determined by an HPLC system (Vanquish, Thermo Scientific, USA) equipped with RefractorMax521 detector (IDEX Health & Science KK, Japan). The chromatographic separation was performed using Rezex RCM-Monosaccharide Ca2+ column (100×7.8 mm, Phenomenex, CA). The column was eluted at 80° C. with water at a flow rate of 0.5m1/min.

The presence of AL39 protein was determined by SDS-PAGE analysis (FIG. 4A). The media samples were separated by SDS-PAGE using methods known in the art (Laemmli, Nature, 227 (5259): 680-685, 1970). The produced AL39 protein was visualized after being stained by staining solution. Produced AL39 was the major protein in the media sample based on SDS-PAGE analysis (FIG. 4A).

After SDS-PAGE and DAE activity screening, the best strain was identified as having high secreted AL39 enzyme production. The produced AL39 enzyme has activity for bioconversion of D-fructose to D-allulose. As shown in FIG. 4A, the produced AL39 protein can be detected in fermentation media by SDS-PAGE analysis, indicating the produced AL39 enzyme can be secreted to the extracellular space of engineered Pichia pastoris strain. The produced enzyme can be easily collected and concentrated from fermentation media after removing the cell pellet by centrifugation. DAE activity can be detected in media sample. As shown in FIG. 4B, around 25% fructose can be converted to allulose after 1 hr in the reaction.

### Example 5: Bioconversion Using Immobilized DAE Enzyme

Enzyme immobilization provides an excellent base for increasing availability of an enzyme to its substrate with greater turnover over a considerable period of time. Described below is a method for efficiently producing immobilized D-allulose epimerase having high activity and excellent durability for bioconversion of fructose to allulose.

AL39 protein was concentrated by filtration. The extracted AL39 was dissolved into 20 mM phosphate buffer (pH 8.0) for immobilization. One unit of DAE activity was defined as the amount of enzyme that catalyzes the formation of 1 μmol D-allulose per min at pH 8.0 and 55° C. The specific activity of immobilized DAE enzyme was defined as the unit per mg enzyme.

The prepared AL39 protein was mixed with water-pre-treated ion exchange resin (LXP-505, SUNRESIN) in 20 mM phosphate buffer (pH 8.0) and slowly stirred in a reactor at 25° C. for 24 hours. Subsequently, the supernatant was removed, and the resulting mixture was washed with 20 mM phosphate buffer (pH 8.0) to obtain an immobilized DAE enzyme.

Immobilized DAE enzyme was added in the reaction mixture for an activity assay. The immobilized enzyme was tested in a 1 ml in vitro reaction system. The reaction system contained 20 mM phosphate buffer, pH 8.0, 1 mM MnSO4, 500 g/L D-fructose. The reaction was performed at 55° C. and the reaction was terminated by heating for 10 min. The samples were analyzed by HPLC. The immobilized enzyme converted more than 23% fructose to allulose after 3 hours.

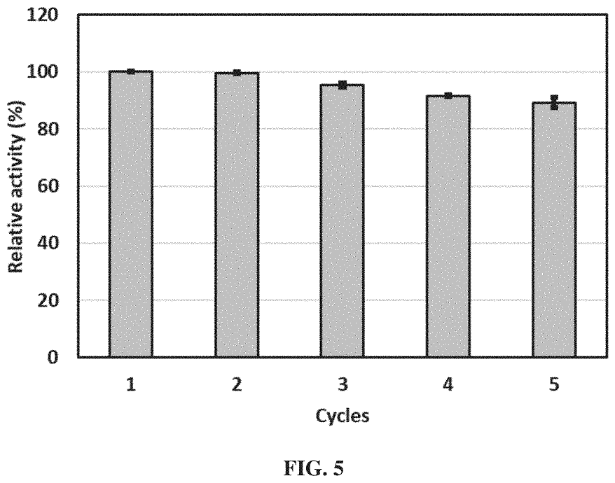

To determine whether AL39 is able to retain activity after use, reuse cycles of immobilized AL39 were tested. 180 mg immobilized AL39 was added in 1 L reaction containing 20 mM phosphate buffer (pH 8.0), 1 mM MnSO4, 500 g/L D-fructose. The reaction was performed at 55° C. After a 5 hour reaction, immobilized enzyme was collected by filtration. The supernatant was analyzed by HPLC to measure allulose production, and the immobilized enzyme was washed with 20 mM phosphate buffer (pH 8.0) 5 times for the next cycle reaction. After 5 cycles, the activity of each cycle was compared. As shown in FIG. 5, the activity of immobilized AL39 only lost 10.76% in the fifth cycle compared to the first cycle, indicating that the immobilized AL39 is stable for allulose bioconversion.

### Reference

1. Lin-Cereghino J, Wong W W, Xiong S, et al. Condensed protocol for competent cell preparation and transformation of the methylotrophic yeast Pichia pastoris. Biotechniques. 2005;38(1):44-48.
2. Laemmli, U. K. (1970). “Cleavage of Structural Proteins during the Assembly of the Head of Bacteriophage T4”. Nature. 227 (5259): 680-685.

TABLE 1
Sequence Information

| | Sequence Description |
|---|---|
| Seq. ID | Internal reference number; GenBank accession number; Source organism; |
| No. | sequence type |
| 1 | AL28; WP_148225901.1; Meiothermus silvanus; amino acid sequence |
| 2 | AL28; WP_148225901.1; Meiothermus silvanus; nucleotide sequence |
| 3 | AL35; VAW22947.1; hydrothermal vent metagenome; amino acid sequence |
| 4 | AL35; VAW22947.1; hydrothermal vent metagenome; nucleotide sequence |
| 5 | AL39; WP_149678132.1; Thermoclostridium caenicola; amino acid sequence |
| 6 | AL39; WP_149678132.1; Thermoclostridium caenicola; nucleotide sequence |
| 7 | AL; 42; VAW20989.1; hydrothermal vent metagenome; amino acid sequence |
| 8 | AL; 42; VAW20989.1; hydrothermal vent metagenome; nucleotide sequence |
| 9 | AL50; WP_119358549.1; Meiothermus granaticius; amino acid sequence |
| 10 | AL50; WP_119358549.1; Meiothermus granaticius; nucleotide sequence |
| 11 | AL51; WP_021097394.1; Rubellimicrobium thermophilum; amino acid |
| | sequence |
| 12 | AL51; WP_021097394.1; Rubellimicrobium thermophilum; nucleotide |
| | sequence |
| 13 | AL66; PJF34855.1; Candidatus Thermofonsia Clade 1 bacterium; amino acid |
| | sequence |
| 14 | AL66; PJF34855.1; Candidatus Thermofonsia Clade 1 bacterium |
| 15 | AL67; WP_103313995.1; Deinococcus koreensis; amino acid sequence |
| 16 | AL67; WP_103313995.1; Deinococcus koreensis; nucleotide sequence |
| 17 | AL74; WP_124032468.1; Olavius algarvensis spirochete endosymbiont; amino |
| | acid sequence |
| 18 | AL74; WP_124032468.1; Olavius algarvensis spirochete endosymbiont; |
| | nucleotide sequence |
| 19 | AL76; WP_029310404.1; WP_029310404.1; amino acid sequence |
| 20 | AL76; WP_029310404.1; WP_029310404.1; nucleotide sequence |
| 21 | AL79; PIY74775.1; Rhodobacterales bacterium; amino acid sequence |
| 22 | AL79; PIY74775.1; Rhodobacterales bacterium; nucleotide sequence |
| 23 | AL105; PZS06712.1; Chloroflexi bacterium; amino acid sequence |
| 24 | AL105; PZS06712.1; Chloroflexi bacterium; |
| 25 | AL106; WP_109678776.1; Spiribacter sp. E85; amino acid sequence |
| 26 | AL106; WP_109678776.1; Spiribacter sp. E85; nucleotide sequence |
| 27 | AL117; WP_101922568.1; Tabrizicola sp. TH137; amino acid sequence |
| 28 | AL117; WP_101922568.1; Tabrizicola sp. TH137; nucleotide sequence |
| 29 | AL118; WP_093362069.1; Tropicimonas isoalkanivorans; amino acid sequence |
| 30 | AL118; WP_093362069.1; Tropicimonas isoalkanivorans; nucleotide sequence |
| 31 | AL119; HBS38151.1; Rhodobacteraceae bacterium; amino acid sequence |
| 32 | AL119; HBS38151.1; Rhodobacteraceae bacterium; nucleotide sequence |
| 33 | AL120; WP_008029474.1; Rhodobacter sp. SW2; amino acid sequence |
| 34 | AL120; WP_008029474.1; Rhodobacter sp. SW2; nucleotide sequence |
| 35 | AL121; PKP63497.1; Alphaproteobacteria bacterium HGW- |
| | Alphaproteobacteria-8; amino acid sequence |
| 36 | AL121; PKP63497.1; Alphaproteobacteria bacterium HGW- |
| | Alphaproteobacteria-8; nucleotide sequence |
| 37 | AL122; WP_037279949.1; Rubellimicrobium mesophilum; amino acid |
| | sequence |
| 38 | AL122; WP_037279949.1; Rubellimicrobium mesophilum; nucleotide sequence |
| 39 | AL123; OHC57906.1; Rhodobacterales bacterium |
| | RIFCSPHIGHO2_02_FULL_62_130; amino acid sequence |
| 40 | AL123; OHC57906.1; Rhodobacterales bacterium |
| | RIFCSPHIGHO2_02_FULL_62_130; nucleotide sequence |
| 41 | AL124; HCW85615.1; Rhodobacterales bacterium |
| | RIFCSPHIGHO2_02_FULL_62_130; amino acid sequence |
| 42 | AL124; HCW85615.1; Rhodobacterales bacterium |
| | RIFCSPHIGHO2_02_FULL_62_130; nucleotide sequence |
| 43 | AL39: Codon optimzed; nucleotide sequence |

Sequence Information
Protein; Meiothermus silvanus
SEQ ID NO: 1
MPRFGAHAFIWAAEWNPEAAEKVIQGATRVGLDFVEIPLLHPESFDVELT
RRLLNGYGVGCTCSLGLPREASLPEHPEAATRFLIQALNVAHQIGSEVLT
GVTYATLGTLSGRAPREADYQAVVKALKPAARHAAALGMRLGIEPVNRYE
TYLINLAAQGLELIRRLDEPNVFLHLDTYHMNIEEKGFRGPIVEAGAHLG
YIHLSESDRGTPGTGNVHWDAVFAGLREIGFSGDLVMESFVALNPDIARA
TCMWRDVVGDPQALVQEGLAFLRGKASEYGLLG;
DNA; Meiothermus silvanus
SEQ ID NO: 2
ATGCCACGCTTCGGAGCTCATGCGTTCATTTGGGCCGCTGAGTGGAACCC
GGAAGCGGCAGAGAAAGTGATTCAGGGTGCAACCCGTGTTGGCCTCGACT
TCGTTGAAATTCCGCTCTTGCACCCGGAATCCTTCGATGTCGAGCTCACA
CGTCGTTTACTAAACGGGTACGGCGTCGGTTGTACGTGTTCTCTGGGCTT
GCCGCGTGAAGCGTCCCTGCCGGAGCACCCCGAAGCCGCGACTCGTTTTC
TGATTCAGGCCCTGAATGTTGCACATCAGATTGGCTCAGAGGTGCTGACC
GGGGTAACCTACGCGACTTTAGGCACTTTATCTGGCCGGGCGCCGCGCGA
AGCTGATTATCAAGCAGTCGTAAAAGCACTGAAACCGGCGGCTAGACATG
CAGCAGCACTGGGTATGCGCCTCGGCATTGAACCCGTTAATCGTTATGAA
ACTTATTTAATTAACCTGGCCGCTCAGGGCCTGGAGTTAATCCGTCGTCT
GGACGAACCGAATGTGTTCCTGCACCTGGATACGTACCACATGAATATTG
AAGAGAAAGGTTTCCGGGGCCCGATCGTGGAAGCGGGCGCCCACCTGGGG
TATATTCATCTGAGCGAGAGTGATCGCGGCACCCCTGGAACGGGCAACGT
CCATTGGGACGCGGTGTTCGCGGGGCTGCGTGAAATTGGCTTCTCAGGCG
ATTTGGTCATGGAAAGCTTCGTCGCACTGAACCCTGATATTGCGCGCGCT
ACATGCATGTGGCGGGACGTCGTTGGCGATCCCCAGGCGCTGGTCCAGGA
AGGCTTAGCATTTCTCCGTGGCAAGGCTAGCGAGTATGGCCTACTCGGTt
aa;
Protein; hydrothermal vent metagenome
SEQ ID NO: 3
MEGFGVHTSMWTMSWDKKGAEYAVSQAVRYQMDFLEIALLSPLDVDAQHS
RRLLEKNHMRAICSLGLPEGAWLSNNPQAGVEYLKIALEKTAEMGCEALS
GVIYGGIGERTGFPPTEKELDNVVRALGEAASYAKSLGLLLGIEPVNRYE
SHLINTGRQSVEMIEKVGASNMFVHLDTYHMNIEEKGVAQGILDAREHIK
YIHLSESDRGTPGAGTCDWDEIFAVLAAINFKGGLAMESFVNMPPEIAYG
LSIWRPVAKSADEVMENGLPFLRNKARQYQLL;
DNA; hydrothermal vent metagenome
SEQ ID NO: 4
ATGGAAGGTTTTGGAGTGCACACGAGCATGTGGACTATGAGTTGGGATAA
GAAGGGTGCGGAGTATGCTGTAAGCCAGGCGGTGCGCTATCAGATGGATT
TTCTTGAAATCGCTCTGCTGAGTCCACTGGATGTCGATGCCCAGCATAGT
CGCCGTCTGCTAGAGAAGAACCACATGCGCGCGATTTGCAGCCTTGGGTT
ACCGGAAGGTGCGTGGCTCAGTAATAACCCTCAGGCCGGAGTGGAATACC
TGAAGATCGCCCTGGAAAAGACTGCGGAGATGGGTTGCGAAGCGCTGTCC
GGCGTAATCTACGGCGGCATTGGCGAAAGAACAGGTTTTCCGCCGACGGA
GAAAGAGCTGGACAATGTCGTGCGCGCGCTGGGTGAAGCGGCATCTTACG
CAAAATCGTTAGGCCTTCTGCTCGGTATTGAACCGGTGAACCGGTATGAG
TCTCATCTGATTAATACGGGACGTCAGAGTGTGGAAATGATTGAAAAGGT
CGGTGCCTCTAACATGTTTGTTCACCTTGACACATACCACATGAATATTG
AAGAAAAGGGTGTGGCACAAGGGATTCTGGACGCGCGCGAACACATAAAA
TATATCCACCTGTCAGAGTCTGACCGAGGGACTCCGGGCGCAGGCACGTG
TGACTGGGATGAGATTTTCGCAGTCCTGGCCGCAATTAACTTTAAAGGCG
GCTTAGCGATGGAATCGTTCGTGAACATGCCGCCGGAAATTGCCTATGGT
CTGTCCATTTGGCGTCCGGTGGCTAAATCGGCTGATGAAGTCATGGAAAA
CGGCCTGCCGTTTCTACGCAACAAAGCGCGCCAGTATCAGCTGCTGtaa;
Protein; Thermoclostridium caenicola
SEQ ID NO: 5
MKYGIFYAYWEKEWKGDFITYIEKVKKLGFDILEVGCGDFHKQPDSYFHT
LRDAAREYDIILTGGYGPRAEHNLCSPDTAVVENALAFYSDIFRKMEIAG
IRSIGGGLYAYWPVDYSREPDKAGDLERSIKNMRRLADIAERHGITLNME
VLNRFEGYLINDTNEGLAYIRAVDKPNVKLMLDTFHMNIEEDSFTEPILQ
AGKYLGHVHVGEPNRKPPREGRIPWGEIGKALRQIGYDGPVVMEPFVTMG
GQVGKDICVWRDLSQGATEEDLDRDAEKSLAFLKGMFEA;
DNA; Thermoclostridium caenicola
SEQ ID NO: 6
ATGAAATATGGTATATTTTACGCATATTGGGAGAAAGAATGGAAAGGCGA
CTTTATCACATACATTGAGAAAGTTAAGAAATTAGGCTTTGACATTTTGG
AAGTCGGCTGCGGTGATTTTCATAAACAGCCGGATTCATACTTCCACACC
CTGCGTGATGCCGCTCGCGAATACGACATTATTCTGACCGGCGGCTATGG
GCCGCGCGCCGAACACAACTTGTGTAGCCCGGATACAGCGGTCGTTGAAA
ATGCCCTGGCATTTTACTCCGATATATTTCGCAAAATGGAAATTGCCGGC
ATCCGTTCGATCGGCGGCGGTCTATATGCGTATTGGCCAGTTGATTACAG
CCGTGAACCCGACAAAGCCGGCGATTTAGAACGTTCCATTAAGAACATGC
GTCGCTTAGCCGATATTGCGGAACGGCATGGTATCACGCTGAATATGGAA
GTGCTCAATCGCTTCGAAGGTTACCTTATTAATGATACCAATGAAGGTCT
GGCCTACATTCGTGCCGTCGATAAACCCAACGTAAAGTTGATGCTGGATA
CATTTCACATGAACATTGAGGAAGATAGCTTTACCGAACCCATTCTCCAG
GCTGGTAAGTACCTGGGCCATGTACATGTCGGCGAACCAAATCGTAAACC
ACCACGGGAAGGTCGTATTCCGTGGGGCGAGATTGGCAAAGCGCTGCGCC
AGATTGGGTACGACGGTCCCGTGGTGATGGAACCGTTTGTTACCATGGGC
GGCCAAGTGGGGAAAGACATTTGTGTGTGGCGGGACCTTAGCCAAGGTGC
GACCGAAGAAGATCTGGATCGCGATGCTGAGAAAAGCCTGGCCTTTCTGA
AAGGAATGTTTGAAGCCtaa;
Protein; hydrothermal vent metagenome
SEQ ID NO: 7
MNLPAKKMVLGVHTFAVAPFWDLEVMRHEARRLKSHGVGLLEIPLLRPEE
INIKATRAFAREFGFELVTSLGLPSNIDAVEDPQSALAFLEPAFKVAAEI
GSNMLSGVTYAPIGKISGQPVTQREKDGICRFLQQAAALAANHGLKLGVE
PCNRYETHLMNTAAQAVEYVEAVAAENLFIHLDTYHMNIEEESFAAGFAK
AAPYLGYVHLSESNRGVPGRAMINWDAVMGALADIGYHGALTLESMNYVD
PDIATALAVWRPVAKNPDDVIDFGLAFLLKAAADAGLTFG;
DNA; hydrothermal vent metagenome
SEQ ID NO: 8
ATGAACCTGCCGGCTAAGAAGATGGTCCTGGGAGTCCATACGTTCGCAGT
CGCCCCGTTTTGGGATCTTGAAGTAATGCGCCACGAGGCCCGCCGTTTAA
AGAGCCATGGCGTAGGGTTATTGGAAATCCCACTTCTCCGGCCAGAAGAA
ATTAATATTAAAGCGACCCGCGCATTTGCTCGTGAATTTGGGTTTGAATT
AGTGACCAGTTTAGGCCTGCCTTCGAATATCGACGCTGTAGAAGATCCGC
AGTCTGCTTTAGCCTTTCTGGAACCGGCGTTCAAAGTGGCTGCTGAAATT
GGTAGCAATATGCTTTCAGGCGTTACCTATGCTCCGATTGGAAAGATAAG
TGGTCAGCCGGTGACCCAGCGTGAGAAGGATGGTATTTGTCGGTTCCTAC
AACAAGCGGCTGCGTTGGCCGCGAACCATGGGTTGAAACTGGGCGTAGAG
CCTTGTAATCGCTATGAAACGCATCTGATGAACACAGCAGCCCAAGCAGT
TGAATACGTTGAGGCTGTGGCAGCGGAAAACCTTTTCATCCACTTAGATA
CCTACCACATGAACATCGAAGAAGAAAGTTTTGCAGCAGGGTTTGCGAAA
GCGGCGCCTTATCTGGGGTACGTGCATCTGAGCGAGAGCAATCGCGGCGT
ACCGGGCCGCGCCATGATCAATTGGGATGCAGTGATGGGTGCCCTTGCCG
ACATCGGGTATCATGGGGCACTGACTCTGGAGTCTATGAATTATGTGGAT
CCGGATATTGCGACTGCCCTTGCGGTCTGGCGTCCGGTTGCCAAGAACCC
GGACGATGTGATAGACTTTGGCCTGGCGTTTCTGCTGAAGGCCGCAGCGG
ATGCGGGTTTGACATTCGGTtaa;
Protein; Meiothermus granaticius
SEQ ID NO: 9
MARFGAHAFIWSADWTPQAAEKVAAGAAAAGLDFVEIPLLRPEAFDSTLT
RRLLEHHGLGCTCSLGLPTEAALPDHPQAAARFLIQALDVAHQMGSPVLS
GVTYATLGALSGRPPTEADYETLAKTLKPVAQHAARLGMRLGLEPVNRYE
TYLINLGSQALDLIQRIGEPNVFVHLDTYHMNIEEKGFKNPIVTVGKHLG
YIHLSESDRGTPGSGNVHWDEVFSGLQAIGFQGDLVMESFVALNPDIARA
TCMWRDVVGDPKTLVHDGLAFLRGKAREYGLL;
DNA; Meiothermus granaticius
SEQ ID NO: 10
ATGGCGCGCTTTGGTGCGCATGCTTTTATTTGGAGTGCTGATTGGACCCC
TCAAGCCGCCGAAAAGGTCGCAGCGGGAGCAGCGGCGGCGGGACTGGATT
TTGTCGAAATACCGCTTCTTCGTCCAGAGGCGTTCGATTCGACACTTACC
CGTAGGTTATTAGAGCATCATGGACTGGGATGCACCTGCTCTCTGGGTCT
GCCTACCGAGGCAGCACTCCCGGACCATCCTCAAGCAGCAGCCCGTTTTC
TCATCCAGGCCTTGGATGTAGCCCATCAAATGGGCAGCCCGGTCCTGAGT
GGGGTCACCTACGCAACCCTGGGCGCACTGTCAGGCCGCCCGCCGACCGA
GGCCGATTATGAAACCCTGGCGAAAACACTGAAGCCAGTAGCGCAGCACG
CCGCCCGCCTGGGTATGCGACTCGGGCTTGAACCGGTTAACCGCTATGAA
ACATACCTGATAAACTTAGGGTCACAGGCGCTGGATCTCATCCAACGTAT
CGGCGAACCGAACGTGTTCGTACACCTGGATACTTATCACATGAACATCG
AAGAGAAAGGTTTTAAGAACCCGATTGTTACCGTCGGTAAACATCTTGGT
TATATTCACCTGTCTGAAAGCGACCGTGGTACCCCAGGTAGCGGTAATGT
GCATTGGGATGAAGTGTTCTCCGGCCTGCAAGCAATCGGTTTTCAGGGTG
ACCTTGTGATGGAGTCGTTTGTGGCACTGAATCCGGATATTGCCCGCGCC
ACGTGCATGTGGCGGGATGTTGTGGGAGATCCGAAAACACTGGTGCATGA
TGGCCTAGCTTTCCTGCGCGGTAAAGCTCGCGAATACGGACTGCTGtaa;
Protein; Rubellimicrobium thermophilum
SEQ ID NO: 11
MQGFGVHTSMWTMHWDRAGAERTIPAAAAYKMDFIEIALLDTAIVDAAHT
RALLEKHGLRAVCSLGLPEPVWASVNPEGAIAHLKRALDKTAEMGAEALS
GVTYGGIGQRTGVPPTPQEYDNIARALEAAAKHAKALGLAFGIEPVNRYE
NHLINTGRQAVEMIEKVGADNIFIHLDTYHMNIEEKGVANGILDAREHLR
YIHLSESDRGTPGEGTCDWDEIFAALAAIGFKGGLAMESFINMPPQVAYG
LAVWRPVAESFEEVMDRGLPFLRNKARQYRLIA;
DNA; Rubellimicrobium thermophilum
SEQ ID NO: 12
ATGCAAGGTTTCGGCGTACACACTAGCATGTGGACGATGCATTGGGATCG
CGCGGGCGCCGAGCGCACCATTCCGGCCGCCGCCGCCTACAAAATGGACT
TTATCGAAATTGCGCTGCTGGATACAGCTATAGTCGATGCAGCGCACACC
CGTGCGCTGCTGGAGAAGCACGGCCTGCGTGCAGTTTGTTCACTCGGATT
ACCGGAGCCGGTCTGGGCCTCTGTGAACCCGGAAGGGGCCATTGCTCACC
TGAAACGCGCGCTGGACAAGACCGCAGAGATGGGAGCTGAAGCTCTGTCT
GGTGTGACTTACGGCGGTATCGGCCAGCGCACAGGCGTACCACCAACGCC
CCAAGAATACGATAACATTGCCCGTGCCCTTGAAGCAGCTGCGAAACATG
CCAAAGCCTTAGGCCTGGCCTTTGGTATCGAACCGGTTAACAGGTATGAA
AACCATTTGATAAACACCGGACGTCAAGCTGTAGAAATGATCGAAAAGGT
CGGGGCAGATAACATTTTCATACACCTGGATACGTATCACATGAATATCG
AAGAGAAAGGTGTGGCAAACGGTATCCTGGATGCCCGAGAACACCTTCGT
TATATTCACCTGTCAGAAAGCGATCGCGGCACTCCTGGCGAAGGGACGTG
TGATTGGGACGAAATCTTCGCTGCGTTGGCCGCCATCGGTTTTAAAGGCG
GCCTTGCTATGGAATCCTTTATTAACATGCCGCCTCAAGTCGCTTACGGC
CTGGCGGTGTGGCGTCCTGTTGCCGAATCTTTTGAAGAAGTCATGGACCG
TGGCCTGCCGTTTCTACGTAACAAGGCTCGTCAGTATAGATTGATCGCTt
aa;
Protein; Candidatus Thermofonsia Clade 1
bacterium
SEQ ID NO: 13
MPTFGAHAFVWIGDWTTESGNYAIAQAGALGFDFIEIPLLAPQRFDAASH
RQALAQAGIQATCSLVLPKGAHMPRYPERARQFLYEALEKVEAVGSQYLG
GCIAYELGYLTGQPPTPEERQVVVEVLRDVAAEARRRGIQLALEACNRYE
TYLYNTLADVRETVLAVGAPNLKLHADTYHMNIEEEGFAQPLIACADVLD
YIHMSESHRGLVGSGNVNWAQVWQALAAIRFNGKLVLESFAAINPDLQAA
TCLWRPPNQPPEVLAREGLRFLREGAAQAQLP;
DNA; Candidatus Thermofonsia Clade 1
bacterium
SEQ ID NO: 14
ATGCCAACCTTCGGCGCCCATGCATTTGTGTGGATTGGAGATTGGACGAC
AGAATCGGGAAACTATGCGATCGCGCAAGCCGGCGCACTGGGCTTCGACT
TCATTGAGATTCCGCTTTTGGCACCGCAGCGTTTTGATGCCGCTTCCCAC
CGTCAAGCCCTGGCGCAGGCCGGCATTCAGGCGACGTGCAGCCTGGTATT
GCCAAAAGGCGCTCACATGCCCAGGTATCCAGAACGTGCGCGTCAGTTCT
TGTATGAAGCGTTAGAAAAGGTAGAGGCGGTTGGAAGTCAATACTTGGGC
GGTTGCATCGCGTACGAACTTGGGTATCTTACCGGTCAGCCACCGACCCC
GGAAGAGCGTCAGGTTGTTGTGGAAGTGCTGCGCGATGTAGCCGCAGAAG
CACGCCGTCGCGGCATTCAGTTGGCACTGGAAGCATGCAATCGCTATGAG
ACTTACCTGTACAACACGCTGGCGGACGTGCGCGAAACGGTTTTAGCGGT
GGGCGCGCCAAACTTGAAGCTGCATGCGGATACCTACCACATGAACATCG
AAGAAGAAGGCTTTGCCCAGCCGCTTATTGCCTGTGCCGACGTGCTGGAT
TATATCCACATGTCGGAATCCCATCGCGGCCTGGTCGGAAGCGGCAACGT
TAATTGGGCGCAGGTCTGGCAAGCGCTCGCCGCAATTCGCTTCAATGGCA
AACTGGTGCTGGAATCGTTTGCCGCGATTAACCCGGACCTTCAGGCCGCT
ACTTGTCTGTGGCGCCCGCCCAACCAGCCGCCGGAAGTGCTGGCCCGCGA
AGGACTGCGATTTCTCCGAGAAGGTGCGGCACAGGCCCAACTACCGtaa;
Protein; Deinococcus koreensis
SEQ ID NO: 15
MLKFGAHAFCWEGDWTDEIGDRVIEQAARAGLDFIEIPLLHPETFDARRH
RRHLEAVGLACVSSLGLPRDAHMPHEPEKAVTFLTGVLDRMEELGARDLT
GCTGYSIGVLTGQGPTSQELDRMVDGLARVTEDARSRGIGVGLEAINRYE
TYMVNTLDDALAVVNRVGSDNLRVHADTYHMNIEETNLREALGRVKGKLN
FIHMSESHRGLVGTGTVPWEDVWQGLADIEFSGYLTLESFAAPNAELAAA
TCIWKPPRHSGQELAQGGLAFLREGATRHGLM;
DNA; Deinococcus koreensis
SEQ ID NO: 16
ATGTTGAAGTTCGGCGCGCACGCCTTTTGCTGGGAAGGCGATTGGACCGA
TGAAATTGGCGATCGCGTCATCGAGCAAGCGGCGCGGGCCGGATTGGATT
TCATTGAAATCCCGCTCTTGCACCCTGAAACCTTCGATGCGCGCCGTCAT
CGTCGCCATCTTGAGGCTGTGGGCCTGGCGTGCGTTTCCAGCCTGGGCCT
TCCGCGTGACGCCCACATGCCACATGAACCGGAGAAAGCGGTAACATTCC
TTACCGGCGTACTGGATCGCATGGAAGAACTGGGCGCTCGCGACCTTACC
GGTTGTACCGGTTATAGCATTGGCGTACTGACCGGCCAGGGTCCGACCTC
TCAAGAGCTCGATCGTATGGTGGATGGTCTGGCTCGTGTGACTGAAGATG
CACGGTCGCGCGGGATTGGCGTAGGCCTTGAAGCCATCAATCGTTATGAA
ACTTATATGGTAAACACGCTGGACGATGCGTTAGCGGTCGTGAATCGTGT
CGGGAGTGACAATCTTCGCGTTCATGCCGATACATACCACATGAACATAG
AAGAAACCAACCTGCGTGAGGCGCTTGGGCGAGTTAAGGGTAAGCTGAAC
TTCATTCACATGAGTGAAAGTCATCGTGGTCTGGTTGGTACCGGGACAGT
TCCATGGGAAGATGTTTGGCAGGGTCTGGCGGATATTGAGTTCAGCGGGT
ATCTTACTCTTGAATCGTTTGCAGCTCCGAATGCCGAGTTGGCGGCAGCT
ACCTGTATCTGGAAACCACCACGGCATAGCGGCCAGGAACTGGCGCAGGG
TGGCCTAGCTTTTCTGCGCGAGGGCGCGACTCGCCACGGCTTAATGtaa;
Protein; Olavius algarvensis spirochete
endosymbiont
SEQ ID NO: 17
MKFGAHAFVWEPEWNDTTSRRVISEAARIGLDFVEIPLLRPERFDGAATK
VLLDEHAVGATYSLGLPSDKSLPERPYLAEPFLRSAIDAIESAGGDTLTG
VLYGTLGELPGRPPNEKDYKVIAQVLRSVADYAKDRGIKLGIEPVNRYET
FLVNTAEQAITLLDRIESDNVFIHLDTYHVNIEEDSFGAAIRLAGDRLGY
IHLSESHRGTPGKGTVDWDDVFGALSDIGFAGPLVMESFVKLNADIARAT
CMWRDIVKDPEALIRDGIAFLEGKAKGYGLF;
DNA; Olavius algarvensis spirochete
endosymbiont
SEQ ID NO: 18
ATGAAATTTGGTGCGCATGCTTTCGTCTGGGAACCAGAATGGAATGATAC
CACCTCGCGCCGTGTGATTTCAGAGGCCGCGCGCATCGGCCTGGATTTTG
TGGAAATACCGCTCCTTCGCCCAGAACGCTTTGACGGCGCCGCTACCAAA
GTCTTGCTGGATGAACATGCCGTGGGCGCCACTTATTCATTGGGCCTGCC
CAGTGACAAAAGCCTGCCAGAACGCCCGTATCTGGCTGAACCCTTCTTAC
GGTCTGCAATCGACGCCATTGAATCCGCAGGCGGTGATACACTGACAGGC
GTGCTGTATGGCACTCTGGGTGAGCTGCCGGGCCGCCCGCCGAACGAAAA
GGACTACAAAGTGATTGCGCAGGTATTACGTAGTGTTGCTGATTACGCGA
AAGACCGTGGCATCAAACTGGGCATTGAGCCGGTAAACCGTTATGAAACC
TTTCTGGTGAATACCGCAGAGCAGGCGATCACATTGTTAGACCGTATCGA
AAGCGACAATGTCTTTATTCATCTGGATACCTATCACGTGAACATCGAAG
AAGATAGTTTTGGCGCGGCTATCCGCCTGGCTGGGGATCGTTTAGGCTAC
ATCCACCTGTCCGAAAGCCACCGTGGCACGCCAGGCAAGGGTACTGTGGA
TTGGGACGACGTTTTCGGAGCGCTTTCCGATATTGGTTTCGCAGGCCCTC
TTGTGATGGAAAGCTTTGTCAAGCTGAATGCAGACATTGCACGTGCGACC
TGCATGTGGCGTGATATTGTGAAAGACCCAGAAGCCCTGATTCGCGATGG
TATTGCGTTTCTCGAGGGGAAAGCGAAAGGCTATGGTCTGTTTtaa;
Protein; Acidiphilium
SEQ ID NO: 19
MKGFGIHTSLWAHDWTEQAARLAIPEAAKHGLAFVEIALIEPDRAETEVT
RGLLEQHGLAACCSLGLPEEARPTTNPDKALEFVTLALEKTAAIGASLFT
GVTYGSIGERTGQPPTAAELDAVARFLDKAAAVARGFGIIFGIEVVNRYE
SHLFNTTEQAVALIERVGAPNIVLHLDTYHMNIEATGQANAIRAAGAHLA
YIHLSESHRGVPGTGTIAWDEVFAGLAGLGFTGGMALESFIHMPPRLAAA
LSVWRPVAPSRAAIIDEGLPFLRNKARQYGLI;
DNA; Acidiphilium
SEQ ID NO: 20
ATGAAAGGCTTTGGCATTCATACAAGTCTGTGGGCCCATGACTGGACCGA
GCAAGCGGCTCGTTTAGCAATTCCGGAAGCGGCAAAACATGGCCTGGCGT
TTGTCGAAATTGCTTTAATTGAACCAGATCGCGCGGAAACTGAAGTTACC
CGCGGGCTGCTGGAACAGCACGGCTTGGCTGCCTGCTGCTCTCTGGGCTT
ACCTGAGGAAGCGCGGCCAACGACGAACCCGGATAAAGCGTTGGAGTTCG
TCACTTTAGCTCTGGAAAAGACCGCGGCGATAGGGGCTAGCCTGTTTACC
GGTGTGACCTACGGGAGCATTGGCGAACGTACGGGCCAGCCACCTACTGC
CGCCGAACTTGATGCAGTAGCAAGGTTCTTGGATAAGGCTGCCGCAGTAG
CGCGCGGTTTCGGTATCATTTTCGGTATCGAAGTAGTGAATCGGTACGAG
TCTCATCTGTTTAACACCACCGAACAGGCCGTTGCACTGATCGAACGTGT
CGGCGCACCGAACATAGTGCTGCACTTAGACACGTATCACATGAACATCG
AAGCCACGGGCCAGGCCAACGCAATACGCGCGGCGGGTGCACATCTGGCC
TATATTCACCTGAGTGAATCGCACAGGGGTGTTCCGGGCACAGGGACCAT
CGCCTGGGACGAGGTGTTTGCAGGTCTTGCCGGCCTGGGCTTTACCGGCG
GGATGGCCCTCGAATCATTTATTCACATGCCGCCCCGCCTGGCCGCCGCG
CTGAGCGTCTGGCGTCCAGTTGCACCGAGTCGCGCGGCCATCATCGATGA
AGGCCTTCCATTTCTGCGAAATAAAGCCCGTCAATACGGCCTCATTtaa;
Protein; Rhodobacterales bacterium
SEQ ID NO: 21
MLSLGLHALAAAPEWRPDLWAAILPRMAAHGVSVIEIPLLRPAELDIAGT
RALAAKHDVELVCSLGLPATLNVAERPDEAFDFIRVALEVTASAGATALS
GVTFGVIGQTTGAAPTTREIDAMTRHVSRSAALAKKLGLRFGIEPCNRYE
THLLNTGAAARAVIERSGADNAFIHLDTYHMNIEEVSHAQGFADAGDLLG
YVHLSESNRGVPGRGTVDWANVFQGLKAAGFDGCAALESFVFVDADLASG
LAIWRPVAENPDDVIDVGFPFLRTAGEAAGLRWAR;
DNA; Rhodobacterales bacterium
SEQ ID NO: 22
ATGCTTTCTCTGGGACTGCATGCGCTTGCAGCCGCCCCTGAATGGCGCCC
GGATCTGTGGGCGGCGATCCTGCCGCGTATGGCGGCCCACGGTGTTAGCG
TTATCGAAATTCCGCTGCTCCGTCCTGCTGAACTGGATATTGCGGGCACG
CGGGCTTTAGCGGCTAAACATGATGTTGAACTCGTTTGTTCATTGGGGCT
GCCAGCCACCCTCAATGTTGCGGAACGCCCTGATGAGGCGTTTGATTTTA
TCCGCGTGGCGTTAGAAGTGACCGCGAGCGCGGGTGCGACCGCGCTGTCA
GGAGTGACATTTGGCGTGATTGGGCAGACGACGGGCGCCGCCCCAACCAC
GCGCGAAATCGATGCTATGACCCGGCATGTGAGCCGTAGCGCAGCCCTCG
CCAAGAAACTGGGCTTGCGCTTTGGCATTGAGCCGTGCAACCGCTACGAA
ACCCACTTACTGAATACTGGCGCAGCCGCTCGGGCCGTGATAGAACGCTC
AGGCGCTGATAACGCCTTCATCCATTTGGATACATATCACATGAATATCG
AAGAAGTGTCTCATGCTCAGGGTTTTGCAGATGCGGGCGACCTGCTGGGA
TATGTGCACTTGTCAGAAAGCAATCGAGGCGTCCCGGGGCGTGGTACCGT
CGATTGGGCGAATGTATTTCAGGGTCTGAAAGCCGCCGGATTCGATGGTT
GCGCCGCACTCGAAAGTTTCGTGTTCGTTGACGCGGACTTAGCATCCGGA
CTTGCCATCTGGCGTCCTGTTGCTGAAAACCCAGATGACGTGATTGACGT
AGGTTTCCCGTTCTTGCGGACAGCCGGAGAGGCGGCGGGCTTGCGCTGGG
CGCGTtaa;
Protein; Chloroflexi bacterium
SEQ ID NO: 23
MVLFGAHTFIWSAEWNPETAEHVIDGAARAGLDFVEIPLLHPDQMDAGGT
RRLLENYGLQCTCSLGLPREAHLPFAPDKATGFLEQAVDVTSDLGSPVLT
GCLYTHLGTLTGKPPTDEEIDLVVRVLKRIARYAQDRGISLGIEPVNRYE
TYLLNLAEQASALLDRIDEANVFVHLDTYHMNIEEKGFYTPIVDTGPRLQ
YIHLSESDRGIPGTGNVHWDEVFRGLKAVKYEGRLVMESFAAVNEDLMGA
TAMWRDVVGDPDRLVTEGLAFLRGKAIEYGLL;
DNA; Chloroflexi bacterium
SEQ ID NO: 24
ATGGTACTGTTTGGGGCGCATACCTTTATTTGGAGCGCCGAATGGAACCC
AGAAACGGCGGAACATGTAATTGATGGCGCAGCTCGCGCCGGCCTGGATT
TCGTTGAGATTCCGTTGCTGCATCCCGATCAGATGGACGCAGGCGGCACG
CGCCGGCTTTTGGAAAACTATGGTCTGCAATGCACATGTAGCTTGGGCCT
GCCGCGCGAAGCGCATCTGCCATTCGCCCCTGACAAGGCAACAGGCTTTC
TGGAACAGGCCGTCGATGTGACAAGTGACCTGGGTAGCCCTGTTTTGACC
GGCTGTTTATATACCCACTTAGGAACGCTGACAGGAAAGCCGCCTACCGA
CGAAGAGATTGATTTGGTCGTTCGTGTCCTGAAGCGAATTGCGCGCTACG
CCCAGGACCGAGGGATTAGTCTGGGCATCGAACCGGTCAACCGCTATGAA
ACCTATCTTCTGAATCTGGCGGAGCAAGCGTCTGCACTGCTCGATCGTAT
TGACGAAGCCAATGTATTTGTGCATCTGGATACCTATCACATGAACATTG
AAGAAAAGGGCTTTTATACTCCGATCGTTGATACCGGGCCGCGTTTACAG
TACATTCACCTGTCCGAATCGGACCGCGGTATCCCGGGTACTGGCAACGT
TCATTGGGACGAGGTGTTTCGAGGCCTCAAGGCCGTTAAATACGAAGGCC
GTCTTGTGATGGAATCTTTCGCCGCAGTCAACGAAGATCTGATGGGTGCA
ACGGCGATGTGGCGGGATGTGGTGGGTGATCCGGATCGATTAGTCACGGA
AGGCCTGGCGTTCTTACGTGGAAAGGCGATTGAGTACGGCCTGCTGtaa;
Protein; Spiribacter sp. E85
SEQ ID NO: 25
MAEFGAHAFIWESDWNPASARRVIAGAAAAGLDFVEIPLLRPESMDTAGT
RRLLAEHRLGVRCSLGLPPAASLPAHPQAAEAFLCRALDVTRALGGPVLT
GVIYGTLGQLPGHPPRPGDLDIVAQTLRRVAAYAADQGLALGIEPVNRYE
THLVNLTDQALELLDAIGADNVFLHLDTYHMNVEEKGFRGPVEAAGKRLR
YIHLSESDRGTPGTGNVHWDEVFDGLAAIGYRGDLVMESFAAVNEDIARA
TCIWRQVAPDPDTLVREGLAFLRGKATARGLIP;
DNA; Spiribacter sp. E85
SEQ ID NO: 26
ATGGCGGAGTTCGGTGCACATGCTTTCATTTGGGAGTCTGACTGGAATCC
GGCGTCAGCTCGCCGTGTCATTGCCGGCGCCGCTGCCGCCGGCCTAGATT
TCGTCGAGATCCCACTGCTCCGCCCGGAATCCATGGACACCGCGGGAACC
CGCAGATTACTTGCTGAACATCGCCTCGGCGTGCGCTGTTCACTGGGCCT
GCCGCCGGCAGCGAGTCTACCCGCTCATCCGCAGGCCGCAGAAGCATTCC
TTTGCCGCGCCCTAGACGTTACGCGTGCGTTGGGCGGACCCGTACTCACT
GGTGTCATCTATGGCACTCTGGGCCAGTTACCGGGTCACCCGCCACGCCC
TGGCGATCTTGACATTGTCGCACAGACCTTACGGCGCGTGGCAGCGTACG
CCGCAGATCAGGGTCTGGCCCTGGGCATTGAACCAGTGAACCGTTATGAA
ACACATTTAGTGAATCTCACGGATCAAGCCCTAGAACTGTTAGATGCGAT
TGGCGCCGACAATGTATTCCTGCATCTGGATACCTATCACATGAACGTGG
AAGAGAAAGGTTTTCGTGGTCCAGTTGAAGCAGCTGGTAAACGTTTGAGA
TACATTCATCTTTCGGAGTCAGATCGTGGTACCCCGGGGACAGGCAACGT
TCACTGGGATGAAGTATTCGACGGTCTGGCTGCAATCGGATACCGTGGGG
ATCTGGTGATGGAAAGTTTTGCGGCCGTGAATGAGGACATTGCGCGTGCG
ACCTGCATCTGGCGTCAAGTTGCCCCAGACCCGGACACTCTGGTTAGAGA
AGGCTTGGCGTTTCTTCGCGGCAAAGCGACCGCCCGCGGCCTTATCCCTt
aa;
Protein; Tabrizicola sp. TH137
SEQ ID NO: 27
MEGFGVHTSMWTMHWDRAGAERTIPAAAAYRMDFIEIALLNTAIVDAAHT
RALLERHGMRAVCSLGLPERNWASVNPEGAIAHLCDCIDTAAAMGAEALS
GVTYGGIGQRTGLPPTMAEYDNIARALAAVAKHAKARGIAFGIEPVNRYE
NHLINTAAQAKWMIEKVGADNIFIHLDTYHMNIEEKGAGNGILDARDHLR
YIHLSESDRGTPGEGTCDWDEVYATLAAIGFKGGLAMESFINMPPEVAYG
LAVWRPVAENFEEVMDKGLPFLRNKARQYRLI;
DNA; Tabrizicola sp. TH137
SEQ ID NO: 28
ATGGAAGGATTTGGTGTACATACTTCCATGTGGACAATGCACTGGGATCG
CGCGGGCGCTGAACGTACCATTCCTGCCGCAGCTGCGTACCGCATGGATT
TCATAGAGATTGCTTTATTAAACACAGCCATTGTTGACGCGGCACACACG
AGGGCTCTCCTTGAACGGCATGGCATGCGCGCAGTGTGTTCATTGGGCTT
ACCTGAACGCAATTGGGCGAGCGTGAACCCGGAAGGCGCCATCGCACATC
TGTGCGATTGTATTGACACAGCAGCAGCCATGGGCGCCGAAGCCCTATCC
GGCGTCACGTATGGCGGCATCGGGCAACGTACGGGGCTCCCGCCTACGAT
GGCCGAATACGATAACATTGCCCGCGCTCTGGCAGCTGTGGCGAAACATG
CTAAAGCACGCGGCATTGCGTTTGGTATCGAACCCGTGAATCGCTACGAA
AATCACTTGATTAATACCGCCGCACAAGCTAAATGGATGATCGAAAAGGT
TGGGGCTGATAATATATTTATTCATCTGGACACCTATCACATGAACATAG
AGGAAAAGGGAGCCGGCAATGGGATCCTCGATGCGCGTGATCATTTGAGA
TATATCCACTTATCGGAATCGGATCGCGGCACTCCGGGCGAAGGCACGTG
TGACTGGGATGAGGTATATGCTACCCTGGCCGCCATTGGTTTTAAAGGCG
GTTTGGCTATGGAATCGTTTATTAATATGCCACCGGAAGTGGCCTATGGT
CTTGCCGTCTGGCGTCCGGTGGCGGAAAATTTTGAGGAAGTGATGGATAA
AGGACTCCCGTTCTTGCGTAATAAAGCGCGCCAATATCGCCTGATAtaa;
Protein; Tropicimonas isoalkanivorans
SEQ ID NO: 29
MKGFGVHTSMWTMHWDRDGAERTIPATAAYGMDFVEIALLDTSIVDAAHT
RALLEKHELRAVCSLGLPEGSWPSVAPEAAVAHLKDVFETAAAMGAEAVS
GVTYGGIGQRSGVPPTEEEYDNVARALQQAAAYAKRLGLAFGIEPVNRYE
NHLINTAWQARDMIEKVGSDNIFIHLDTYHMNIEEKGVANGILDARDHLR
YIHLSESDRGTPGEGCCDWNEVFGTLSAIGFEGGMAMESFINMPPQIAYG
LAVWRPVADSFEDVMDRGLPFLRNKARQYRLA;
DNA; Tropicimonas isoalkanivorans
SEQ ID NO: 30
ATGAAGGGTTTCGGGGTGCACACGAGTATGTGGACGATGCATTGGGACCG
GGATGGCGCGGAACGTACAATTCCAGCGACGGCCGCGTATGGCATGGATT
TTGTCGAGATCGCTCTGTTGGATACCTCTATCGTTGATGCTGCGCACACC
CGGGCGCTCCTGGAGAAACACGAGCTGCGCGCCGTTTGCTCGTTGGGCCT
GCCGGAAGGCTCGTGGCCTTCGGTTGCCCCAGAGGCGGCGGTAGCGCATC
TGAAAGACGTATTTGAAACCGCAGCCGCGATGGGCGCCGAAGCGGTTTCG
GGCGTCACATACGGCGGTATTGGCCAACGTTCCGGTGTGCCGCCCACCGA
GGAAGAATATGACAACGTAGCTCGTGCCCTGCAACAGGCAGCGGCTTACG
CCAAACGCTTAGGCTTAGCCTTTGGCATTGAACCTGTGAACCGCTACGAA
AACCATTIGATCAACACTGCATGGCAGGCCCGTGACATGATTGAAAAGGT
AGGCTCGGACAATATTTTCATCCATTTAGATACTTACCACATGAATATAG
AAGAGAAAGGTGTGGCGAACGGTATCTTAGACGCACGTGATCACCTTCGT
TATATTCATCTTTCAGAGAGCGACCGTGGCACTCCGGGTGAAGGTTGCTG
CGATTGGAACGAAGTTTTCGGGACCTTGAGCGCTATTGGGTTTGAAGGTG
GTATGGCGATGGAATCGTTTATTAACATGCCGCCGCAGATAGCTTACGGC
CTGGCCGTTTGGCGACCAGTTGCGGACTCCTTCGAAGATGTGATGGATCG
CGGGCTGCCGTTTCTGCGTAACAAAGCGCGTCAGTACCGTCTGGCTtaa;
Protein; Rhodobacteraceae bacterium
SEQ ID NO: 31
MQGFGVHTSMWTMHWDRLGAERTIPAAAAYKMDFIEIALLDTSVVDAHHT
RDLLAKHEMRAVCSLGLPRESWASVNPDGAIAHLIDAMDYTKEMGGEALS
GVTYGGIGERSGVPPTEAEYDNIARALEVAAKYAKTLGIAFGIEPVNRYE
SHLINTSWQAKEMIDKIGADNIFIHLDTYHMNIEEKGAGNGILAAREHLR
YIHLSESDRGTPGEGTCDWDEIFATLAAVEFKGGLAMESFINMPPQVGYG
LGIWRPVANSFEEVMDKGLPFLRNKAAQYRLI;
DNA; Rhodobacteraceae bacterium
SEQ ID NO: 32
ATGCAAGGTTTTGGTGTCCATACCAGTATGTGGACAATGCACTGGGATCG
CCTGGGCGCGGAACGAACCATTCCGGCCGCGGCTGCGTACAAAATGGACT
TTATTGAAATTGCTCTTCTGGACACCAGCGTCGTGGATGCGCATCACACT
CGTGATCTTCTGGCGAAACATGAAATGCGTGCGGTTTGCTCGCTGGGCTT
GCCTCGCGAATCTTGGGCCAGTGTTAACCCGGATGGTGCCATAGCTCACC
TTATAGATGCAATGGACTATACTAAAGAGATGGGCGGCGAGGCACTGTCT
GGCGTCACCTATGGCGGTATCGGTGAACGTAGCGGTGTTCCGCCGACGGA
AGCTGAATATGATAATATCGCACGTGCCCTAGAAGTTGCCGCCAAATACG
CCAAAACGCTGGGCATTGCCTTTGGCATTGAACCCGTGAACCGCTATGAA
AGCCACCTGATTAATACCAGTTGGCAGGCTAAAGAAATGATTGATAAAAT
CGGTGCAGATAACATCTTCATTCACTTAGATACCTACCACATGAACATTG
AAGAGAAAGGCGCCGGTAATGGAATCCTGGCAGCTCGGGAACACCTTCGC
TACATTCATTTGAGCGAAAGCGACCGGGGTACACCCGGCGAAGGCACCTG
TGATTGGGACGAAATATTTGCGACCCTTGCTGCCGTTGAGTTCAAAGGTG
GCTTGGCCATGGAGAGTTTCATTAATATGCCGCCGCAAGTGGGATATGGC
TTGGGTATCTGGCGCCCGGTCGCCAATTCATTCGAGGAAGTGATGGATAA
AGGCCTGCCGTTCCTGCGTAACAAAGCGGCCCAGTATCGTCTTATTtaa;
Protein; Rhodobacter sp. SW2
SEQ ID NO: 33
MEGFGVHTSMWTMQWDRAGAERTIPAAAAYKMDFIEIALLNTAIVDAPHT
RALLQKHGLRAVASLGLPQQNWASVNPEGAIEQLCRSLDTAAAMGCEALS
GVTYGGIGERSGLPPTMAEYDNVARALAAAAKHAKKLGLAFGIEPVNRYE
SHLINTGWQAKWMIEKVGADNIFIHLDTYHMNIEEKGAGNGILDAREHLR
YVHLSESDRGTPGEGTVDWDEIFATLAAVGFKGGLAMESFINMPPELGYG
LAVWRPVAESFQAVMDKGLPFLRNKARQYRLI;
DNA; Rhodobacter sp. SW2
SEQ ID NO: 34
ATGGAAGGTTTCGGTGTCCATACTTCCATGTGGACCATGCAATGGGACCG
TGCTGGTGCCGAACGGACGATACCAGCCGCTGCGGCATATAAGATGGATT
TCATCGAAATCGCGCTGCTGAATACGGCCATTGTGGATGCCCCGCATACC
CGCGCACTGTTACAGAAACATGGTCTGCGCGCCGTTGCGAGTCTGGGCCT
GCCACAACAGAACTGGGCGAGTGTCAATCCTGAGGGTGCCATTGAGCAGT
TATGCCGCTCACTGGACACCGCTGCCGCGATGGGTTGCGAAGCCCTGTCA
GGGGTTACCTACGGCGGCATCGGAGAGCGCAGCGGTCTGCCGCCAACAAT
GGCAGAATATGACAACGTGGCCCGTGCGTTGGCGGCGGCAGCAAAACACG
CTAAGAAACTGGGCCTGGCGTTTGGAATCGAACCTGTGAACAGATATGAG
TCTCACTTGATCAACACCGGATGGCAAGCAAAATGGATGATTGAAAAGGT
GGGTGCGGATAACATCTTTATCCACCTGGATACTTATCACATGAATATTG
AGGAAAAGGGTGCAGGCAACGGCATTCTCGATGCGCGCGAACACCTGCGT
TACGTGCATCTTAGCGAAAGCGACCGTGGGACCCCTGGAGAAGGTACTGT
TGACTGGGATGAAATATTCGCGACACTCGCCGCAGTTGGATTTAAAGGCG
GATTAGCAATGGAAAGCTTTATCAACATGCCACCGGAACTGGGATATGGC
CTGGCAGTATGGCGCCCGGTGGCGGAATCCTTTCAAGCAGTAATGGATAA
AGGTTTACCTTTTCTGCGGAACAAAGCGCGTCAGTATCGTCTGATCtaa;
Protein; Alphaproteobacteria bacterium
HGW-Alphaproteobacteria-8
SEQ ID NO: 35
MRGFGVHTSMWTMKWDRAGAERAVAAAAHYKMDFIEIALLNAPGVDAPHS
RALLEKHGLRAVCSLGLPEAAWPSRNPEAAVAHLKVALDKTAEMGCEALT
GVTYGGIGERTGLPPSATELDNVARALREAAAHAKKLGLLFGIEPVNRYE
THLINTGRQAVEMIEKVGAENMFIHLDTYHMNIEEKGAANGILDAREHIK
YIHLSESDRGTPGWGTCDWDEIFAVLSAIGFKGGLAMESFINMPPEVAYG
LSVWRPVARDEAEVMDNGLPFLRGKARQYRLI;
DNA; Alphaproteobacteria bacterium
HGW-Alphaproteobacteria-8
SEQ ID NO: 36
ATGCGTGGTTTTGGCGTCCATACATCTATGTGGACTATGAAATGGGACCG
TGCAGGTGCCGAGCGTGCCGTGGCGGCGGCAGCCCACTATAAGATGGACT
TCATCGAAATTGCGCTGCTGAACGCGCCGGGTGTCGATGCACCGCATAGT
CGGGCACTCCTGGAGAAACATGGACTGCGCGCGGTCTGTTCATTGGGGCT
TCCGGAAGCCGCTTGGCCGAGCCGCAACCCGGAAGCCGCCGTTGCTCATC
TGAAAGTGGCGCTCGATAAAACCGCTGAAATGGGTTGTGAAGCGCTGACC
GGAGTCACCTACGGCGGCATCGGGGAACGGACCGGCCTGCCACCGTCTGC
CACCGAACTGGATAACGTGGCACGCGCTTTACGCGAAGCCGCAGCACACG
CTAAGAAGCTGGGGCTCCTCTTCGGCATCGAACCGGTTAACCGCTACGAA
ACCCATCTGATTAACACTGGTCGGCAAGCCGTTGAAATGATTGAGAAAGT
CGGCGCAGAGAATATGTTTATTCACCTGGACACCTATCACATGAACATCG
AGGAAAAGGGCGCAGCGAATGGTATTCTGGATGCACGCGAACACATTAAA
TATATCCATCTTTCAGAATCTGATCGCGGTACCCCGGGCTGGGGTACCTG
CGATTGGGATGAAATTTTCGCAGTGCTGAGCGCAATTGGATTTAAAGGTG
GTTTGGCGATGGAAAGTTTTATTAACATGCCGCCGGAAGTTGCGTATGGC
CTGTCAGTATGGCGACCAGTCGCACGGGATGAAGCGGAAGTTATGGATAA
CGGCCTTCCGTTTCTTCGTGGTAAAGCCCGCCAGTACCGCTTAATTtaa;
Protein; Rubellimicrobium mesophilum
SEQ ID NO: 37
MQGFGVHTSMWTMNWDRAGAERTIPAASAYGMDFIEIALLNAPAVDAPHT
RALLEKHGMRAVCSLGLPERNWASVNPEGAIEHLRQALEVTAALGAEALS
GVTYGGIGQRTGVPPTAGEYDNIARALEAAARYARELGIAFGIEPVNRYE
NHLVNTAAQAKWMIEKVGADNIFIHLDTYHMNIEEKGVGNGILDAREHLR
YIHLSESDRGTPGEGTCDWDEVFATLAAIGFKGGLAMESFINMPPEIGYG
LAVWRPVAESFEEVMDRGLPFLRNKAKQYRLV;
DNA; Rubellimicrobium mesophilum
SEQ ID NO: 38
ATGCAAGGGTTTGGCGTCCACACTTCGATGTGGACCATGAACTGGGATCG
TGCCGGCGCCGAACGTACAATCCCGGCCGCGAGCGCATACGGTATGGATT
TTATTGAAATTGCGCTGCTGAATGCCCCGGCGGTGGACGCTCCACATACG
CGCGCCCTTCTTGAAAAGCACGGGATGCGTGCAGTTTGCAGTTTGGGTTT
GCCTGAGCGTAATTGGGCAAGCGTCAACCCTGAAGGCGCGATCGAGCACC
TTCGACAAGCCCTCGAAGTGACCGCCGCCCTTGGGGCAGAAGCGTTAAGC
GGTGTCACATACGGCGGTATAGGTCAGCGCACAGGGGTGCCACCCACCGC
TGGCGAGTATGATAACATCGCCCGTGCACTGGAAGCCGCTGCACGCTATG
CACGAGAACTGGGTATCGCCTTTGGTATCGAACCGGTTAACCGGTACGAG
AACCATCTGGTGAACACCGCGGCCCAAGCTAAATGGATGATAGAAAAGGT
TGGCGCGGATAATATTTTCATCCATTTGGATACATATCACATGAACATTG
AGGAGAAAGGTGTCGGCAACGGTATCCTGGATGCGCGTGAGCACCTGCGC
TATATTCATCTGTCTGAGAGCGATCGCGGCACACCAGGCGAGGGTACTTG
CGATTGGGACGAAGTATTCGCAACCCTGGCAGCAATTGGTTTCAAAGGCG
GACTTGCTATGGAATCGTTTATCAACATGCCGCCAGAAATTGGATACGGC
TTGGCCGTTTGGCGACCTGTGGCAGAGAGTTTTGAAGAAGTCATGGACCG
CGGCCTGCCGTTCCTGCGGAACAAGGCGAAACAGTACCGTCTTGTGtaa;
Protein; Rhodobacterales bacterium
SEQ ID NO: 39
MEGFGVHTSMWTMHWDRAGAERTIPAAAAYKMDFIEIALLNAAMIDPPHT
RALLEKHNMRAVASLGLPQRNWASVNPDGAAAHLIEAMDVAAAMGAEALS
GVTYGGIGERTGLPPTMAEYDNIARALGQAAKHAKKLGIAFGIEPVNRYE
NHLINTGWQAKWMIEKVGADNIFIHLDTYHMNIEEKGAGNGILDAREHLR
YIHLSESDRGTPGEGTCDWDEVYATLAAIGFKGGLAMESFINMPPEVGYG
LAVWRPVANSFEEVMDKGLPFLRNKARQYRLI;
DNA; Rhodobacterales bacterium
SEQ ID NO: 40
ATGGAAGGCTTCGGTGTGCACACGAGTATGTGGACGATGCACTGGGATCG
TGCTGGTGCTGAACGTACGATTCCGGCCGCCGCCGCGTACAAGATGGACT
TCATTGAAATTGCGCTGCTGAACGCAGCGATGATCGATCCGCCACATACT
CGCGCGCTTCTGGAGAAACATAACATGCGGGCTGTTGCGAGTCTCGGACT
TCCACAACGCAATTGGGCCTCGGTTAATCCAGATGGTGCCGCGGCCCATC
TTATCGAGGCGATGGACGTGGCGGCAGCAATGGGTGCCGAAGCGCTTTCC
GGAGTGACATACGGCGGAATCGGTGAACGCACTGGCCTGCCGCCAACTAT
GGCAGAGTACGATAACATTGCACGAGCACTCGGGCAGGCCGCCAAACATG
CCAAGAAGCTTGGGATCGCTTTCGGCATAGAGCCGGTGAATCGCTATGAG
AATCATCTGATCAACACTGGCTGGCAGGCCAAATGGATGATTGAGAAAGT
GGGTGCTGATAACATCTTTATCCATCTGGATACATATCACATGAATATCG
AGGAAAAGGGCGCTGGTAATGGTATTTTAGATGCACGTGAACACCTGCGT
TATATTCATTTGTCTGAGTCCGATCGCGGCACGCCGGGTGAAGGCACGTG
TGACTGGGATGAAGTTTACGCGACACTGGCAGCCATTGGCTTTAAAGGTG
GACTCGCGATGGAATCATTCATCAACATGCCGCCGGAAGTGGGTTATGGT
CTGGCAGTTTGGCGCCCGGTGGCGAACAGCTTTGAAGAAGTTATGGACAA
AGGTCTGCCGTTTCTCCGCAATAAAGCGCGGCAGTACCGCCTGATCtaa;
Protein; Rhodobacterales bacterium
RIFCSPHIGHO2_02_FULL_62_130>
SEQ ID NO: 41
MEGFGVHTSMWTMKWDREGTERAVQAAVDYKMDFLEIALLDAPSVDAAHT
RKLLEDNDMRAVCSLGLPEAVWPSRDPESAIAFMKGVFDKANEMGAEAVS
GVTYGGIGERTDMPPTEAELSNVARALEACASYAKSLGLRFGIEPVNRYE
THLLNTGWQARDMIERIGSDNIFIHLDTYHMNIEEKGAASGILDAREHLK
YIHLSESDRGTPGEGCCDWDEIFATLAAINFTGGLAMESFINMPPELAHG
LSVWRPVAPDFQAVMDKGLPFLRNKAAQYRLV;
DNA; Rhodobacterales bacterium
RIFCSPHIGHO2_02_FULL_62_130
SEQ ID NO: 42
ATGGAAGGTTTTGGGGTACACACCAGCATGTGGACGATGAAATGGGATCG
TGAGGGCACCGAGCGGGCTGTACAGGCGGCAGTTGATTACAAAATGGATT
TTCTGGAAATTGCTCTGCTTGACGCTCCGAGCGTGGACGCGGCACACACG
CGCAAACTCCTGGAAGATAACGATATGCGTGCGGTGTGCAGTTTAGGGTT
ACCAGAAGCGGTGTGGCCGAGTCGCGACCCAGAGAGCGCAATTGCTTTTA
TGAAAGGAGTATTCGATAAGGCCAACGAAATGGGCGCCGAGGCAGTGAGT
GGGGTCACGTATGGCGGCATAGGCGAACGCACAGACATGCCACCGACAGA
AGCGGAGCTGAGCAACGTAGCCCGCGCACTGGAAGCGTGCGCGAGTTATG
CAAAATCATTAGGTCTGCGATTTGGCATTGAGCCTGTGAATCGTTATGAA
ACCCATCTGCTGAATACCGGTTGGCAAGCACGCGATATGATCGAACGTAT
CGGTTCAGATAACATTTTCATTCATTTGGATACGTACCACATGAATATCG
AAGAGAAAGGCGCGGCCTCTGGTATTTTAGATGCCCGTGAACACTTGAAA
TACATCCATTTATCTGAAAGTGACAGAGGCACGCCAGGCGAAGGTTGTTG
CGACTGGGATGAAATTTTCGCCACCCTCGCCGCCATTAATTTTACCGGCG
GTCTTGCCATGGAAAGTTTCATTAACATGCCGCCAGAGCTGGCGCATGGT
CTGAGCGTGTGGCGCCCCGTGGCCCCGGACTTCCAAGCGGTGATGGATAA
AGGCCTGCCGTTCCTGCGAAACAAAGCCGCGCAGTACCGCCTGGTCtaa;
Protein; Thermoclostridium caenicola,
encoded by codon optimized DNA
sequence SEQ ID NO: 44
SEQ ID NO: 43
KYGIFYAYWEKEWKGDFITYIEKVKKLGFDILEVGCGDFHKQPDSYFHTL
RDAAREYDIILTGGYGPRAEHNLCSPDTAVVENALAFYSDIFRKMEIAGI
RSIGGGLYAYWPVDYSREPDKAGDLERSIKNMRRLADIAERHGITLNMEV
LNRFEGYLINDTNEGLAYIRAVDKPNVKLMLDTFHMNIEEDSFTEPILQA
GKYLGHVHVGEPNRKPPREGRIPWGEIGKALRQIGYDGPVVMEPFVTMGG
QVGKDICVWRDLSQGATEEDLDRDAEKSLAFLKGMFEA;
DNA; Thermoclostridium caenicola,
Codon optimized
SEQ ID NO: 44
AAGTACGGTATCTTCTATGCTTATTGGGAAAAGGAATGGAAGGGTGACTT
CATTACTTACATTGAAAAGGTTAAGAAGCTGGGATTCGATATTCTTGAAG
TTGGTTGTGGTGACTTCCATAAACAACCTGATTCTTACTTCCATACTCTT
AGAGATGCTGCTAGAGAATATGATATTATTCTTACTGGTGGTTACGGTCC
TAGAGCTGAACATAACTTGTGTTCTCCAGATACTGCTGTTGTTGAAAACG
CTCTTGCCTTCTATTCTGATATCTTCAGAAAAATGGAGATCGCTGGTATT
AGATCCATTGGTGGTGGTCTGTATGCTTACTGGCCTGTTGATTATTCTAG
AGAACCTGATAAGGCTGGTGATCTTGAAAGATCCATTAAGAACATGAGAA
GATTGGCTGATATTGCTGAAAGACATGGTATTACTCTTAATATGGAAGTT
CTTAACAGATTCGAAGGTTATCTTATTAACGATACTAACGAAGGTCTTGC
TTATATTAGAGCTGTTGATAAACCAAACGTTAAATTGATGTTGGATACCT
TCCACATGAACATTGAAGAAGATTCCTTCACTGAACCTATTCTTCAAGCT
GGTAAGTATCTTGGTCATGTTCATGTTGGTGAACCTAATAGAAAGCCACC
TAGAGAAGGTAGAATCCCTTGGGGTGAAATTGGTAAAGCTCTTAGACAAA
TTGGTTACGATGGTCCTGTTGTTATGGAACCATTCGTTACTATGGGTGGT
CAAGTTGGTAAGGATATCTGTGTCTGGAGAGACTTATCTCAAGGTGCTAC
TGAAGAGGATCTTGATAGAGATGCTGAAAAGTCTCTTGCCTTCTTGAAAG
GTATGTTCGAAGCT;

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### EQUIVALENTS AND SCOPE

Those skilled in the art will recognize, or be able to ascertain using no more than routine experimentation, many equivalents to the specific embodiments of the present disclosure described herein. The scope of the present disclosure is not intended to be limited to the above description, but rather is as set forth in the appended claims.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub—range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. All references, patents and patent applications disclosed herein are incorporated by reference with respect to the subject matter for which each is cited, which in some cases may encompass the entirety of the document. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of producing allulose, the method comprising contacting a fructose substrate with a reaction mixture comprising:
(a) an enzyme system comprising a D-allulose 3-epimerase enzyme;
(b) a host cell transformed with a recombinant vector comprising a nucleic acid molecule that encodes a D-allulose 3-epimerase enzyme; and/or
(c) a lysate of the host cell of (b), under conditions such that the fructose substrate is converted into allulose, wherein the D-allulose 3-epimerase enzyme comprises an amino acid sequence having at least 80% sequence identity to a polypeptide selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43.

2. The method of claim 1, wherein the reaction mixture comprises a D-allulose 3-epimerase enzyme comprising an amino acid sequence having at least 80% sequence identity to a polypeptide selected from the group consisting of SEQ ID NO:1, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, and SEQ ID NO:25.

3. The method of claim 2, wherein the D-allulose 3-epimerase enzyme comprises an amino acid sequence having at least 90% sequence identity to a polypeptide selected from the group consisting of SEQ ID NO:1, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, and SEQ ID NO:25.

4. The method of claim 2, wherein the D-allulose 3-epimerase enzyme comprises an amino acid sequence having at least 95% sequence identity to a polypeptide selected from the group consisting of SEQ ID NO:1, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, and SEQ ID NO:25.

5. The method of claim 2, wherein the D-allulose 3-epimerase enzyme comprises the amino acid sequence of SEQ ID NO:1, SEQ ID NO:5, SEQ ID NO:7, SEQ ID NO:9, SEQ ID NO:13, SEQ ID NO:15, SEQ ID NO:17, SEQ ID NO:19, SEQ ID NO:21, SEQ ID NO:23, or SEQ ID NO:25.

6. The method of claim 1, wherein the reaction mixture comprises a D-allulose 3-epimerase enzyme comprising an amino acid sequence having at least 90% sequence identity to a polypeptide selected from the group consisting of SEQ ID NO:3, SEQ ID NO:11, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43.

7. The method of claim 6, wherein the D-allulose 3-epimerase enzyme comprises an amino acid sequence having at least 95% sequence identity to a polypeptide selected from the group consisting of SEQ ID NO:3, SEQ ID NO:11, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43.

8. The method of claim 6, wherein the D-allulose 3-epimerase enzyme comprises the amino acid sequence of SEQ ID NO:3, SEQ ID NO:11, SEQ ID NO:27, SEQ ID NO:29, SEQ ID NO:31, SEQ ID NO:33, SEQ ID NO:35, SEQ ID NO:37, SEQ ID NO:39, SEQ ID NO:41, and SEQ ID NO: 43.

9. The method of claim 1, wherein the conditions comprise maintaining the enzyme system and the fructose substrate at a temperature between 25° C. and 75° C.

10. The method of claim 1, wherein the conditions comprise maintaining the enzyme system and the fructose substrate at a pH between 4 and 10.

11. The method of claim 1, wherein the D-allulose 3-epimerase enzyme is in isolated form.

12. The method of claim 1, wherein the D-allulose 3-epimerase enzyme is immobilized on a solid substrate.

13. The method of claim 1, wherein the reaction mixture comprises a host cell transformed with a recombinant vector comprising a nucleic acid molecule that encodes the D-allulose 3-epimerase enzyme or a lysate of said host cell, wherein the nucleic acid molecule encoding the D-allulose 3-epimerase enzyme comprises a polynucleotide sequence having at least 80% sequence identity to a polynucleotide sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40,SEQ ID NO:42, and SEQ ID NO: 44.

14. The method of claim 13, wherein the host cell is selected from the group consisting of a yeast cell, a filamentous fungal cell, a bacterial cell, a mammalian cell, a plant cell, and a Labryinthulomycetes cell.

15. The method of claim 14, wherein the host cell is E. coli or P. pastoris.

16. The method of claim 13, wherein the recombinant vector exists as a self-replicating nucleic acid molecule within the host cell.

17. The method of claim 13, wherein the recombinant vector is integrated into the host cell chromosome.

18. The method of claim 1, further comprising adding at least one type of metal ions to the reaction system, said at least one type of metal ions selected from the group consisting of magnesium ions, manganese ions, copper ions, zinc ions, nickel ions, cobalt ions, iron ions, aluminum ions, and calcium ions.

19. The method of claim 18, wherein the metal ions are added at a concentration from about 0.01 mM to about 5mM.

20. The method of claim 1, comprising removing a product stream comprising allulose from the reaction system as the fructose substrate is converted into allulose.
